(19) United States
(12) Patent Application Publication
(10) Pub. No.: US 2025/0213803 A1
(43) Pub. Date: Jul. 3, 2025

(54) DRY POWDER FORMULATIONS, DOSE CONTAINERS AND INHALERS CONTAINING THE SAME, AND METHODS

(71) Applicant: KINDEVA DRUG DELIVERY L.P., Woodbury, MN (US)

(72) Inventors: Stephen STEIN, Woodbury, MN (US); Leslie NEMBO, Lowell, MN (US)

(21) Appl. No.: 18/981,988

(22) Filed: Dec. 16, 2024

Related U.S. Application Data
(60) Provisional application No. 63/615,087, filed on Dec. 27, 2023.

(51) Int. Cl. A61M 15/00; A61K 31/135; A61K 31/58; A61K 47/24; A61K 47/26

(52) U.S. Cl. CPC A61M 15/0003; A61K 31/135; A61K 31/58; A61K 47/24; A61K 47/26; A61M 15/0051

(57) ABSTRACT

Dry powder formulations (preferably configured for use with a dry powder inhaler) including a first sub-formulation that includes a first drug and a second sub-formulation that includes a second drug, the first sub-formulation and the second sub-formulation being in physical contact.

### CROSS-REFERENCE TO RELATED APPLICATION

This application claims the benefit of U.S. Provisional Patent Application No. 63/615,087, filed Dec. 27, 2024 which is incorporated herein by reference in its entirety.

### INTRODUCTION

The use of dry powder inhalation devices (i.e., dry powder inhalers) in the administration of drugs, for example, in bronchodilation therapy, is well known. Therapies involving combinations of different and complementary drugs are known. These can be formulated as a single combination (multi-active) formulation or as or two or more distinct formulations that are administered in-tandem or sequentially. Although single formulation combination products may offer added convenience for the inhaler user, some drugs are difficult to formulate together in a single formulation. For example, the drugs may chemically interact with each other in an undesirable way when formulated together.

In-tandem and/or sequential administration of two or more separate formulated drugs often involves multiple patient actions and/or a complex inhaler device. For sequential administration, for example, a patient may need to prepare and administer each dose of each drug separately. For in-tandem administration, a single actuation may deliver both formulations; however, the inhaler device is complicated. For example, such an inhaler device may include a mechanism to keep the formulations separate, such as two distinct blister strips, until the two formulations are simultaneously delivered upon use of the device. As such, improvements to combination dry powder inhaler therapy are desirable.

### SUMMARY

The present disclosure describes a dry powder formulation, preferably configured for use with a dry powdered inhaler. The dry powdered formulation includes a first sub-formulation and a second sub-formulation. The first sub-formulation is in physical contact with the second sub-formulation. The first sub-formulation includes a first drug, and the second sub-formulation includes a second drug. The first drug and the second drug are different.

In some embodiments, the first sub-formulation, the second sub-formulation, or both, are excipient free. In some embodiments, the first sub-formulation, the second sub-formulation, or both, include one or more excipients. In some embodiments, the one or more excipients include sugar, a force control agent, a phospholipid, a biocompatible polymer, or any combination thereof. In some embodiments, the one or more excipients includes sugar, and the sugar is present as coarse carrier particles, fine excipient particles, a force control agent, or any combination thereof.

The present disclosure describes a dose container having a formulation of the present disclosure disposed within. The first sub-formulation and the second sub-formulation are in physical contact within the dose container. In some embodiments, the dose container is in the form of a blister strip, a reservoir, or a capsule.

The present disclosure further describes a dry powder inhaler. The dry powder inhaler includes a dose container having a formulation of the present disclosure disposed within.

The present disclosure also describes a method of making a formulation of the present disclosure, a method of making a dose container of the present disclosure, and a method of making a dry powder inhaler of the present disclosure.

All scientific and technical terms used herein have meanings commonly used in the art unless otherwise specified. The definitions provided herein are to facilitate understanding of certain terms used frequently herein and are not meant to limit the scope of the present disclosure.

The term “substantially” as used here has the same meaning as “significantly,” and can be understood to modify the term that follows by at least about 90%, at least about 95%, or at least about 98%. The term “substantially free” of a particular compound means that the compositions of the present invention contain less than 1,000 parts per million (ppm) of the recited compound. The term “essentially free” of a particular compound means that the compositions of the present invention contain less than 100 parts per million (ppm) of the recited compound. The term “completely free” of a particular compound means that the compositions of the present invention contain less than 20 parts per billion (ppb) of the recited compound. In the context of the aforementioned phrases, the compositions of the present invention contain less than the aforementioned amount of the compound whether the compound itself is present in unreacted form or has been reacted with one or more other materials.

The term “not substantially” as used here has the same meaning as “not significantly,” and can be understood to have the inverse meaning of “substantially,” i.e., modifying the term that follows by not more than 25%, not more than 10%, not more than 5%, or not more than 2%.

All numbers are assumed to be modified by the term “about” and in certain embodiments, preferably, by the term “exactly.” As used herein in connection with a measured quantity, the term “about” refers to that variation in the measured quantity as would be expected by the skilled artisan, and is understood to have the same meaning as “approximately” and to cover a typical margin of error, such as +5% of the stated value. At the very least, and not as an attempt to limit the doctrine of equivalents to the scope of the claims, each numerical parameter should at least be construed in light of the number of reported significant digits and by applying ordinary rounding techniques.

Terms such as “a,” “an,” and “the” are not intended to refer to only a singular entity, but include the general class of which a specific example may be used for illustration.

The terms “a,” “an,” and “the” are used interchangeably with the term “at least one.” The phrases “at least one of” and “comprises at least one of” followed by a list refers to any one of the items in the list and any combination of two or more items in the list.

As used here, the term “or” is generally employed in its usual sense including “and/or” unless the content clearly dictates otherwise. The term “and/or” means one or all of the listed elements or a combination of any two or more of the listed elements.

The recitations of numerical ranges by endpoints include all numbers subsumed within that range (e.g., 1 to 5 includes 1, 1.5, 2, 2.75, 3, 3.80, 4, 5, etc. or 10 or less includes 10, 9.4, 7.6, 5, 4.3, 2.9, 1.62, 0.3, etc.). Where a range of values is “up to” or “at least” a particular value, that value is included within the range.

As used here, “have,” “having,” “include,” “including,” “comprise,” “comprising,” or the like are used in their open-ended sense, and generally mean “including, but not limited to.” It will be understood that “consisting essentially of,” “consisting of,” and the like are subsumed in “comprising” and the like. As used herein, “consisting essentially of,” as it relates to a composition, product, method, or the like, means that the components of the composition, product, method, or the like are limited to the enumerated components and any other components that do not materially affect the basic and novel characteristic(s) of the composition, product, method, or the like.

The term “polymer” and “polymeric material” include, but are not limited to homopolymers, copolymers, blends of two or more homopolymers, blends of two or more copolymers, blends of one or more homopolymers and one or more copolymers that have any geometric configuration such as a linear configuration, branched configuration, graft configuration, star configuration, isotactic symmetry, syndiotactic symmetry, atactic symmetry, or any combination thereof. Copolymers are polymers polymerized from two or more monomers and include block copolymers, alternating copolymers, periodic copolymers, statistical copolymers, stereoblock copolymers, gradient copolymers, and the like.

The words “preferred” and “preferably” refer to embodiments that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the disclosure, including the claims.

Any direction referred to here, such as “top,” “bottom,” “left,” “right,” “upper,” “lower,” and other directions and orientations are described herein for clarity in reference to the figures and are not to be limiting of an actual device or system or use of the device or system. Devices or systems as described herein may be used in a number of directions and orientations.

The present disclosure will be described with respect to embodiments and with reference to certain drawings, but the invention is not limited thereto. The drawings described are only schematic and are non-limiting. In the drawings, the size of some of the elements can be exaggerated and not drawn to scale for illustrative purposes.

The above summary of the present disclosure is not intended to describe each disclosed embodiment or every implementation of the present disclosure. The description that follows more particularly exemplifies illustrative embodiments. In several places throughout the disclosure, guidance is provided through lists of examples, which examples may be used in various combinations. In each instance, the recited list serves only as a representative group and should not be interpreted as an exclusive or exhaustive list. Thus, the scope of the present disclosure should not be limited to the specific illustrative structures described herein, but rather extends at least to the structures described by the language of the claims, and the equivalents of those structures. Any of the elements that are positively recited in this specification as alternatives may be explicitly included in the claims or excluded from the claims, in any combination as desired. Although various theories and possible mechanisms may have been discussed herein, in no event should such discussions serve to limit the claimable subject matter.

### BRIEF DESCRIPTION OF DRAWINGS

The present disclosure will be described with respect to embodiments and with reference to certain drawings, but the invention is not limited thereto. The drawings described are only schematic and are non-limiting. In the drawings, the size of some of the elements can be exaggerated and not drawn to scale for illustrative purposes.

FIG. 1 is a block diagram schematic of a dry powder inhaler.

FIG. 2A-2C are schematics of a blister strip dose container (2A), a capsule dose container (2B), and a reservoir dose container (2C).

FIG. 3 is a schematic of a formulation having two distinct sub-formulations.

FIG. 4 is a schematic of a formulation having substantially homogenously intermixed sub-formulations.

FIG. 5 is a schematic of a formulation having partially or heterogeneously intermixed sub-formulations.

FIG. 6 is the mean Aerodynamic particle size distribution (APSD) profile of a commercially available inhaler.

FIG. 7 is the mean APSD profile of a formulation including vilanterol trifenatate (VT; VT; Control in Example 1).

FIG. 8 is the mean APSD profile of a formulation including fluticasone furoate (FTF; FTF Control in Example 1).

FIG. 9 is the mean APSD profile of a first formulation including a VT sub-formulation and a FTF sub-formulation (VF/FTF F1 in Example 1).

FIG. 10 is the mean APSD profile of a second formulation including a VT sub-formulation and a FTF sub-formulation (VF/FTF F2 in Example 1).

FIG. 11 is the mean APSD profile of a third formulation including a VT sub-formulation and a FTF sub-formulation (VF/FTF F3 in Example 1).

FIG. 12 is the mean APSD profile of a fourth formulation including a VT sub-formulation and a FTF sub-formulation (VF/FTF F4 in Example 1).

FIG. 13 is the mean APSD profile of a formulation including mometasone furoate (MF; MT Control in Example 2).

FIG. 14 is the mean APSD profile of a formulation including a VT sub-formulation and a MF sub-formulation (VT/MF F1 Example 2).

### DETAILED DESCRIPTION

Combination bronchodilation therapy, where two or more drugs (also called active ingredients or actives) are administered to a patient at the same time or in quick succession, may be more effective than monotherapy. However, for some drugs it is challenging to formulate them together as a single formulation due to adverse interactions between the drugs. As a result, combination therapy is often accomplished by separate administration of individually formulated drugs. Alternatively, combination therapy is accomplished using an inhaler that keeps the drug formulations physically separated prior to actuation by the user.

The present disclosure provides dry powder inhaler formulations (also called inhaler formulations, dry powder formulations, or formulations) having two or more sub-formulations, each sub-formulation having one or more drugs. The components of each sub-formulation are formulated separately then the sub-formulations are combined to form the dry powder inhaler formulation. Prior to aerosolization, for example, using a dry powdered inhaler, the sub-formulations are in physical contact. Also provided are dose containers that contain a dry powder formulation of the present disclosure. The present disclosure further provides inhalers containing a dry powder formulation of the present disclosure, for example, within a dose container.

The formulations of the present disclosure are distinct from traditional dry powder inhaler single formulations of two or more drugs. Single formulations of two or more drugs are formed when two or more drugs are formulated with the same optional excipients. For example, two or more drugs may be formulated with an excipient such that both of the drugs are associated with the excipient. An example of such a formulation includes first fine drug particles and second fine drug particles commingled on the same carrier particles.

In some cases, some excipients and/or formulation configurations (e.g., ordered blend, combination particles, or spheronized aggregates as described herein) may work better for different drugs. In contrast to traditional dry powder inhaler single formulations of two or more drugs where each drug is associated with the same excipients in the same formulation configuration, the sub-formulations of the present disclosure allow each drug to be first formulated with one or more optional excipients in separate sub-formulations. The individual components of each sub-formulation may enhance the properties of the drug contained within the sub-formulation.

The sub-formulations are combined prior to inclusion in a dry powdered inhaler and prior to use of a dry powdered inhaler containing the formulation by a user. For example, the sub-formulations may be combined in a dose container and the dose container provided to a dry powder inhaler user. In the dry powder inhaler formulation, the sub-formulations are in physical contact prior to aerosolization. For example, in the dry powder inhaler formulation, at the sub-formulations are in physical contact prior to user dose preparation, and user aerosolization of the dry powdered formulation.

The dry powder inhaler formulations of the present disclosure are described as including a first dry powder sub-formulation (also called a first sub-formulation) and a second dry powder sub-formulation (also called a second sub-formulation). The first dry powder sub-formulation includes a first drug. The second dry powder formulation includes a second drug. The first drug and the second drug are different. As such, the first dry powder sub-formulation and the second dry powder sub-formulation include different drugs. In some embodiments, the first dry powder sub-formulation, the second dry powder sub-formulation, or both, include two or more drugs. In such embodiments, the first dry powder sub-formulation and the second dry powder sub-formulation contain at least one different drug.

Each sub-formulation includes at least one drug (e.g., the first drug of the first dry powder sub-formulation or the second drug of the second dry powder sub-formulation). Examples of drug types that may be included in a sub-formulation include bronchodilator drugs (sometimes referred to as bronchodilators), anti-inflammatory drugs, polypeptides, and other types of drugs. In some embodiments, the first drug, the second drug, or both include a bronchodilator, an anti-inflammatory drug, or both. In some embodiments, the first dry powder sub-formulation includes a bronchodilator and the second dry powder sub-formulation includes an anti-inflammatory drug.

Bronchodilators include short-acting beta agonists, intermediate-acting beta agonists, long-acting beta agonists, anticholinergics, long-acting muscarinic agonists, and methylxanthines. Examples of short-acting beta agonists include albuterol, levalbuterol, metaproterenol, terbutaline, epinephrine, a pharmaceutically acceptable salt thereof, a pharmaceutically acceptable ester thereof, or any combination thereof. An example of an intermediate-acting beta agonist is procaterol. Examples of long-acting beta agonists include abediterol, carmoterol, salmeterol, milveterol, formeoterol, indacaterol, arformoterol, vilanterol, olodaterol, a pharmaceutically acceptable salt thereof, a pharmaceutically acceptable ester thereof, or any combination thereof. Examples of anticholinergics include ipratropium, tiotropium, aclidinium, umeclidinium, oxitropium, glycopyrronium, a pharmaceutically acceptable salt thereof, a pharmaceutically acceptable ester thereof, or any combination thereof. Examples of long-acting muscarinic agonists include tiotropium, aclidinium, glycopyrronium, a pharmaceutically acceptable salt thereof, a pharmaceutically acceptable ester thereof, or any combination thereof. Examples of methylxanthines include theophylline, aminophylline, a pharmaceutically acceptable salt thereof, a pharmaceutically acceptable ester thereof, or any combination thereof.

Anti-inflammatory drugs include corticosteroids, antihistamines, mast cell stabilizers, other drugs, or any combination thereof. Other drugs include, for example, ergotamine and cyclosporine. Examples of corticosteroids include beclomethasone, budesonide, ciclesonide, fluticasone, flunisolide, triamcinolone, mometasone, a pharmaceutically acceptable salt thereof, a pharmaceutically acceptable ester thereof, or any combination thereof. Examples of antihistamines include cromolyn sodium, ketotifen, azelastine, a pharmaceutically acceptable salt thereof, a pharmaceutically acceptable ester thereof, or any combination thereof. Examples of mast cell stabilizers include cromolyn sodium, nedocromil sodium, ketotifen, a pharmaceutically acceptable salt thereof, a pharmaceutically acceptable ester thereof, or any combination thereof.

Examples of polypeptides that may be included in a sub-formulation include insulin (e.g., recombinant insulin), calcitonin (e.g., salmon calcitonin), parathyroid hormone, lysozyme, immunoglobulin G (IgG), or any combination thereof.

Examples of other drugs that may be included in a sub-formulation include omalizumab, zileuton, insulin, pentamidine, calcitonin, leuprolide, alpha-I-antitrypsin, interferon, nintedanib, a pharmaceutically acceptable salt thereof, a pharmaceutically acceptable ester thereof, or any combination thereof.

The absolute amount of drug in a formulation is dependent at least in part on the desired dose, the presence or absence of excipients, or both. The relative amount of drug in a sub-formulation may vary. In embodiments, where the sub-formulation does not include an excipient, the sub-formulation includes 100 weight percent (wt-%) of the drug. In some embodiments, where a sub-formulation includes one or more excipients, the sub-formulation includes less than 100 wt-% of the drug. For example, in some embodiments, the first sub-formulation and the second sub-formulation may each independently include 0.01 wt-% to 30 wt-% of drug (e.g., first drug in the first dry powder sub-formulation or second drug in the second dry powder sub-formulation) based on the weight of the sub-formulation. In some embodiments, the first sub-formulation and the second sub-formulation may each independently include 30 wt-% or less, 25 wt-% or less, 20 wt-% or less, 15 wt-% or less, 10 wt-% or less, 5 wt-% or less, 1 wt-% or less, 0.5 wt-% or less, 0.1 wt-% or less, or 0.01 wt-% or less of drug based on the weight of the sub-formulation. In some embodiments, the first sub-formulation and the second sub-formulation may each independently include 0.01 wt-% or greater, 0.1 wt-% or greater, 0.5 wt-% or greater, 1 wt-% or greater, 5 wt-% or greater, 10 wt-% or greater, 15 wt-% or greater, 20 wt-% or greater, or 25 wt-% or greater of drug based on the weight of the sub-formulation. In embodiments when a formulation includes one or more excipients, the first sub-formulation and the second sub-formulation may each independently include 10 wt-% or less, 5 wt-% or less, or 1 wt-% or less of drug based on the weight of the sub-formulations.

In some embodiments, the drug of a dry powder sub-formulation may be formulated with one or more excipients. An excipient is any non-drug component of a sub-formulation. Stated differently, a dry powder sub-formulation may include one or more excipients. In other embodiments, the drug of a dry powder sub-formulation may be formulated without an excipient. Stated differently, a dry powder sub-formulation may be excipient free. In some embodiments, the first dry powder sub-formulation includes one or more excipients, and the second dry powder sub-formulation includes one or more excipients. In some embodiments, the first dry powder sub-formulation includes one or more excipients, and the second dry powder sub-formulation is excipient free. Examples of excipients include sugars, force control agents, phospholipids, biocompatible polymers, or any combination thereof.

In some embodiments, a sugar functions as a force control agent. In some embodiments, a sugar does not function as a force control agent.

In some embodiments, biocompatible polymer functions as a force control agent. In some embodiments, a biocompatible polymer does not function as a force control agent.

In embodiments where a sub-formulation includes one or more excipients, the total amount of excipients (total excipients) in the sub-formulation may vary. In some embodiments, the first sub-formulation and the second sub-formulation may each independently include 0.01 wt-% to 99.9 wt-% total excipients based on the weight of the sub-formulation. In some embodiments, the first sub-formulation and the second sub-formulation may each independently include 0.01 wt-% or greater, 0.1 wt-% or greater, 1 wt-% or greater, 10 wt-% or greater, 20 wt-% or greater, 30 wt-% or greater, 40 wt-% or greater, 50 wt-% or greater, 60 wt-% or greater, 70 wt-% or greater, 80 wt-% or greater, 90 wt-% or greater, or 95 wt-% or greater total excipients based on the weight of the sub-formulation. In some embodiments, the first sub-formulation and the second sub-formulation may each independently include 99.9 wt-% or less, 95 wt-% or less, 90 wt-% or less, 80 wt-% or less, 70 wt-% or less, 60 wt-% or less, 50 wt-% or less, 40 wt-% or less, 30 wt-% or less, 20 wt-% or less, 10 wt-% or less, 5 wt-% or less, or 1 wt-% or less total excipients based on the weight of the sub-formulation.

A sub-formulation may include one or more sugars. The term sugar includes any natural sugar or derivative thereof such as a sugar alcohol or sugar alcohol polymer. Examples of sugars include lactose (e.g., lactose monohydrate), trehalose, sucrose, mannitol, glucose, or any combination thereof. The total amount of sugar (total sugar) in a sub-formulation may vary. In some embodiments, the first sub-formulation and the second sub-formulation may each independently include 0.1 wt-% to 99.9 wt-% total sugar based on the weight of the sub-formulation. In some embodiments, the first sub-formulation and the second sub-formulation may each independently include 0.1 wt-% or greater, 1 wt-% or greater, 10 wt-% or greater, 20 wt-% or greater, 30 wt-% or greater, 40 wt-% or greater, 50 wt-% or greater, 60 wt-% or greater, 70 wt-% or greater, 80 wt-% or greater, 90 wt-% or greater, or 95 wt-% or greater total sugar based on the weight of the sub-formulation. In some embodiments, the first sub-formulation, the second sub-formulation, or both may each independently include 99.9 wt-% or less, 95 wt-% or less, 90 wt-% or less, 80 wt-% or less, 70 wt-% or less, 60 wt-% or less, 50 wt-% or less, 40 wt-% or less, 30 wt-% or less, 20 wt-% or less, 10 wt-% or less, 5 wt-% or less, or 1 wt-% or less total sugar based on the weight of the sub-formulation.

In some embodiments, a sub-formulation may include fine particles For example, the first sub-formulation, the second sub-formulation, or both may include fine particles. Fine particles include a compound of a single chemical identity and have a volume-weighted median diameter (D₅₀) of 10 micrometers (μm) or less as measured according to the Particle Size Test Method. In some embodiments, fine particles have a D₅₀ of 5 μm or less, 4.5 μm or less, 4 μm or less, 3.5 μm or less, 3 μm or less, 2.5 μm or less, 2 μm or less, 1.5 μm or less, 1 μm or less, or 0.5 μm or less as measured according to the Particle Size Test Method.

Fine particles may be obtained through micronization; that is, the breaking down of larger particles into fine particles. For example, larger particles may be reduced in particles size through milling such as jet milling or ball milling or through exposing the particles to high shear conditions in a liquid medium. Alternately, fine particles may be formed by spray drying, spray freeze drying, supercritical fluid technology, or the like. In some embodiments, fine particles may be conditioned to minimize amorphous content of the fine particle.

A sub-formulation may include fine drug particles. Stated differently, a drug may be present in a sub-formulation as fine drug particles. In some embodiments, the first sub-formulation includes fine drug particles. In some embodiments, the second sub-formulation includes fine drug particles. In some embodiments, the first sub-formulation and the second sub-formulation include fine drug particles.

A sub-formulation may include fine excipient particles. Stated differently, in some embodiments an excipient may be present in a sub-formulation as fine excipient particles. For example, in some embodiments, the first dry powder sub-formulation, the second dry powder sub-formulation, or both include fine excipient particles. Fine excipient particles may increase the flowability of fine drug particles. In embodiments where the sub-formulation includes a sugar excipient, the sugar may be in the form of a fine particle. In some embodiments, the first sub-formulation, the second sub-formulation, or both, include lactose as fine excipient particles. In some embodiments, a sub-formulation that includes fine drug particles may also include fine excipient particles.

In some embodiments, the first sub-formulation and the second sub-formulation may each independently include 0.1 wt-% to 90 wt-% fine excipient particles based on the weight of the sub-formulation. In some embodiments, the first sub-formulation and the second sub-formulation may each independently include 0.1 wt-% or greater, 1 wt-% or greater, 2 wt-% or greater, 3 wt-% or greater, 4 wt-% or greater, 5 wt-% or greater, 10 wt-% or greater, 15 wt-% or greater, or 20 wt-% or greater, 30 wt-% or greater, 40 wt-% or greater, 50 wt-% or greater, 60 wt-% or greater, 70 wt-% or greater, or 80 wt-% or greater fine excipient particles based on the weight of the sub-formulation. In some embodiments, the first sub-formulation and the second sub-formulation may each independently include 90 wt-% or less, 80 wt-% or less, 70 wt-% or less, 60 wt-% or less, 50 wt-% or less, 40 wt-% or less, 30 wt-% or less, 25 wt-% or less, 20 wt-% or less, 15 wt-% or less, 10 wt-% or less, 5 wt-% or less, 4 wt-% or less, 3 wt-% or less, 2 wt-% or less, or 1 wt-% or less fine excipient particles based on the weight of the sub-formulation.

In some embodiments, a sub-formulation may include coarse carrier particles. Stated differently, in some embodiments an excipient may be present in a sub-formulation as coarse carrier particles. For example, in some embodiments, the first dry powder sub-formulation, the second dry powder sub-formulation, or both may include coarse carrier particles. Coarse carrier particles include a compound of a single chemical identity and have a volume-weighted median diameter (D₅₀) of 50 μm to 200 μm as measured by the Particle Size Test Method. In some embodiments, the coarse carrier particles may have an average particle size of 50 μm or greater, 60 μm or greater, 70 μm or greater, 80 μm or greater, 90 μm or greater, 100 μm or greater, 125 μm or greater, 150 μm or greater, or 175 μm or greater as measured by the Particle Size Test Method. In some embodiments, the coarse carrier particles may have a D₅₀ of 200 μm or less, 175 μm or less, 150 μm or less, 125 μm or less 100 μm or less, 90 μm or less, 80 μm or less, 70 μm or less, or 60 μm or less as measured by the Particle Size Test Method. In embodiments where the sub-formulation includes a sugar excipient, the sugar may be in the form of a coarse carrier particles. For example, an example of coarse carrier particles is lactose, such as lactose monohydrate. In some embodiments, the first sub-formulation, the second sub-formulation, or both, include lactose as coarse carrier particles.

In some embodiments, to improve fine drug particle flowability, fine drug particles may be formulated with coarse carrier particles. For example, to prevent and/or reduce fine particle agglomeration, fine drug particles may be formulated with coarse carrier particles. The fine drug particles may adhere to the larger coarse carrier particles forming coarse carrier particle-fine drug particle complexes. Upon patient inhalation using a dry powder inhaler, the fine drug particles can be liberated from the coarse carrier particles by the shear force and turbulence generated by the airflow in the inhaler and in the upper respiratory tract. The freed fine drug particles may be able to reach the desired treatment location.

In some embodiments, the first sub-formulation and the second sub-formulation may each independently include 40 wt-% to 99.5 wt-% coarse carrier particles based on the weight of the sub-formulation. In some embodiments, the first sub-formulation and the second sub-formulation may each independently include 40 wt-% or greater, 50 wt-% or greater, 60 wt-% or greater, 70 wt-% or greater, 80 wt-% or greater, 90 wt-% or greater, or 95 wt-% or greater coarse carrier particles based on the weight of the sub-formulation. In some embodiments, the first sub-formulation and the second sub-formulation may each independently include 99.5 wt-% or less, 95 wt-% or less, 90 wt-% or less, 80 wt-% or less, 70 wt-% or less, 60 wt-% or less, or 50 wt-% or less coarse carrier particles based on the weight of the sub-formulation.

In some embodiments, a sub-formulation includes fine drug particles, coarse carrier particles, and fine excipient particles. In some such embodiment, the fine excipient particles may increase the ability of the fine drug particles to be released from the coarse carrier particles.

In some embodiments, a sub-formulation may include a force control agent. For example, in some embodiments, the first sub-formulation, the second sub-formulation, or both, may include a force control agent. A force control agent may function to decrease interparticle interactions of fine drug particles with other fine drug particles or with coarse carrier particles (if present in the sub-formulation). As such, a force control agent may prevent aggregation of fine drug particles. Examples of force control agents include magnesium stearate, mannitol, leucine (e.g., L-leucine), glucose, polyethylene glycol, or any combination thereof. In some embodiments, the first sub-formulation, the second sub-formulation, or both include magnesium stearate.

In some embodiments, the first sub-formulation and the second sub-formulation may each independently include 0.1 wt-% to 5 wt-% of a force control agent based on the weight of the sub-formulation. In some embodiments, the first sub-formulation and the second sub-formulation may each independently include 0.1 wt-% or greater, 0.5 wt-% or greater, 0.7 wt-% or greater, 1 wt-% or greater, 2 wt-% or greater, 3 wt-% or greater, or 4 wt-% or greater of a force control agent based on the weight of the sub-formulation. In some embodiments, the first sub-formulation and the second sub-formulation may each independently include 5 wt-% or less, 4 wt-% or less, 3 wt-% or less, 2 wt-% or less, 1 wt-% or less, 0.7 wt-% or less, or 0.5 wt-% or less of a force control agent based on the weight of the sub-formulation. Preferably, in some embodiments, the first sub-formulation and the second sub-formulation may each independently include 0.5 wt-% to 2 wt-% of a force control agent based on the weight of the sub-formulation. Most preferably, in some embodiments, the first sub-formulation and the second sub-formulation may each independently include 0.5 wt-% to 1 wt-% of a force control agent based on the weight of the sub-formulation.

In some embodiments, a sub-formulation may include a phospholipid. For example, in some embodiments, the first sub-formulation, the second sub-formulation, or both may include a phospholipid. A phospholipid may function as an emulsifier at an oil/water interface for during spray drying formulation techniques. The inclusion of a phospholipid during this technique may allow for the creation of porous particles. Phospholipid that may be present in a sub-formulation include a phosphatidylcholine. Examples of phosphatidylcholine include distearoylphosphatidylcholine (DSPC), dipalmitoylphosphatidylcholine (DPPC), or both.

In some embodiments, a sub-formulation may include a biocompatible polymer. For example, in some embodiments, the first sub-formulation, the second sub-formulation, or both may include a biocompatible polymer. The biocompatible polymer may facilitate drug release in the lungs. Examples of biocompatible polymers include natural polymers such as albumin, carrageenan, chitosan, gelatin, hyaluronic acid, and any combination thereof. Examples of biocompatible polymers also include synthetic polymers such as poly(lactic acid), oligo(lactic acid), poly(vinyl alcohol), poly(ethylene glycol) poly(lactic-co-glycolic acid), poly(lactic acid)-poly(ethylene glycol)-poly(lactic acid), or any combination thereof. In some embodiments, a biocompatible polymer may be present as a force control agent. For example, poly(ethylene glycol) may be present in a sub-formulation as a force control agent.

Dry-powder sub-formulations may have a variety of configurations. The type of dry-powder formulation configuration is dependent at least in part on the presence or absence of one or more excipients. Specifically, drugs formulated with (i.e., the sub-formulation includes) one or more excipients may be configured as an ordered blend, combination particles, or spheronized aggregates. Drugs formulated without (i.e., the sub-formulation does not include) one or more excipients may be configured as spheronized aggregates, or engineered particles having a particular morphology.

In some embodiments, the first sub-formulation, the second-sub formulation, or both are configured as an ordered blend. An ordered blend sub-formulation configuration includes fine drug particles and coarse carrier particles. In an ordered blend, the fine drug particles adsorb onto the surface of the excipient carrier particles. Ordered blends may include excipients in addition to the coarse carrier particles. For example, an ordered blend may include fine excipient particles (such as those described herein), a force control agent (such as those described herein), or both. Ordered blend configurations can be formed by mixing fine drug particles with coarse carrier particles and any optional additional excipients.

A sub-formulation configured as an ordered blend may include one or more excipients in any suitable amount such as those amounts described herein, and include a drug in any suitable amount such as those amounts described herein. For example, a sub-formulation configured as an ordered blend may include 0.01 wt-% to 30 wt-% drug, such as 0.01 wt-% to 10 wt-%, 0.5 wt-% to 10 wt-%, 1 wt-% to 10 wt-% or 1 wt-% to 5 wt-% drug based on the weight of the sub-formulation. In some embodiments, a sub-formulation configured as an ordered blend may include 40 wt-% to 99. 5 wt-% coarse carrier particles, such as 40 wt-% to 80 wt-% or 40 wt-% to 60 wt-% coarse carrier particles based on the weight of the sub-formulation. In some embodiments, a sub-formulation configured as an ordered blend may include 1 wt-% to 30 wt-% fine excipient particles, such as 1 wt-% to 15 wt-% or 2 wt-% to 10 wt-% fine excipient particles based on the weight of the sub-formulation. In some embodiments, a sub-formulation configured as an ordered blend may include 0.1 wt-% to 5 wt-% force control agent, such as 0.5 wt-% to 2 wt-% or 0.5 wt-% to 1 wt-% force control agent based on the weight of the sub-formulation.

In some embodiments, the first sub-formulation, the second-sub formulation, or both are configured as combination particles. Each combination particle includes a network of drug molecules and excipient molecules. The drug molecules and excipient molecules may be homogeneously or heterogeneously distributed throughout the combination particle. Examples of excipients that may be included in combination particles include sugars (such as those described herein), a biocompatible polymer (such as those described herein), a phospholipid (such as those described herein), a force control agent (such as those described herein), or any combination thereof.

A sub-formulation configured as combination particles may include one or more excipients in any suitable amount such as those amounts described herein, and include a drug in any suitable amount such as those amounts described herein. For example, a sub-formulation configured as combination particles may include 10 wt-% or less, 5 wt-% or less, or 1 wt-% or less drug based on the weight of the sub-formulation. In some embodiments, a sub-formulation configured as combination particles may include 90 wt-% or greater or 95 wt-% or greater total excipients based on the weight of the sub-formulation. In some embodiments, a sub-formulation configured as combination particles may include 50 wt-% or greater, 60 wt-% or greater, 70 wt-% or greater, 80 wt-% or greater, 90 wt-% or greater, or 95 wt-% or greater total sugar based on the weight of the sub-formulation. In some embodiments, a sub-formulation configured as an combination particles may include 0.1 wt-% to 5 wt-% force control agent such as 0.5 wt-% to 2 wt-% or 0.5 wt-% to 1 wt-% force control agent based on the weight of the sub-formulation.

Combination particles may have a volume-weighted median diameter D₅₀ of 10 μm or less as measured by the Particle Size Test Method. In some embodiments, the combination particles of a sub-formulation may have a D₅₀ of 10 μm or less, 5 μm or less, 4 μm or less, 3 μm or less, 2 μm or less, 1 μm or less, or 0.1 μm or less as measured by the Particle Size Test Method. In some embodiments, the combination particles of a sub-formulation have a D₅₀ of 0.01 μm or greater, 0.1 μm or greater, 1 μm or greater, 2 μm or greater, 3 μm or greater, 4 μm or grater, or 5 μm or greater as measured by the Particle Size Test Method.

A sub-formulation configured as combination particles may be formed, for example, by spray drying a solution that includes the drug and one or more excipients. A sub-formulation configured as combination particles may also be formed, for example, by dehydrating (e.g., through lyophilization) a solution that includes the drug and one or more excipients. During the formation process, drug molecules and excipient molecules come together in a single particle.

In some embodiments, the first sub-formulation, the second-sub formulation, or both are configured as spheronized aggregates. A spheronized aggregate consists of fine drug particles, or includes fine drug particles and fine excipient particles. In a spheronized aggregate, the fine drug particles and fine excipient particles (if present) aggregate together to form an agglomerate. In embodiments where the spheronized aggregate is excipient free, the fine drug particles may be primarily crystalline (i.e., 90% or greater crystalline). In embodiments, where the spheronized aggregate includes fine drug particles and fine excipient particles, the fine drug particles and the fine excipient particles may be homogeneously or heterogeneously distributed throughout a spheronized aggregate. Examples of excipients that may be included in a combination particle configuration include fine sugar particles.

A sub-formulation configured as spheronized aggregates may include one or more excipients in any suitable amount such as those amounts described herein, and include a drug in any suitable amount such as those amounts described herein. In embodiments where the spheronized aggregate is excipient free, the spheronized aggregate includes 100 wt-% drug. In some embodiments where a sub-formulation is configured as spheronized aggregates that include fine excipient particles, the sub-formulation may include 10 wt-% to 30 wt-% of drug such as 10 wt-% to 20 wt-% or 5 wt-% to 15 wt-% drug based on the weight of the sub-formulation. In some embodiments where a sub-formulation is configured as spheronized aggregates that include fine excipient particles, the sub-formulation may include 70 wt-% to 90 wt-% fine excipient particles such as 70 wt-% to 80 wt-% fine excipient particles based on the weight of the sub-formulation.

Spheronized aggregates may have a volume-weighted median diameter D₅₀ of 0.1 millimeter (mm) to 2 mm as measured by the Particle Size Test Method. In some embodiments, the spheronized aggregates of a sub-formulation may have a D₅₀ of 0.1 mm or greater, 0.2 mm or greater, 0.3 mm or greater, 0.5 mm or greater, 0.7 mm or greater, 1 mm or greater, or 1.5 mm or greater as measured by the Particle Size Test Method. In some embodiments, the spheronized aggregates of a sub-formulation may have a D₅₀ of 2 mm or less, 1.5 mm or less, 1 mm or less, 0.7 mm or less, 0.5 mm or less, 0.4 mm or less, 0.3 mm or less, or 0.2 mm or less as measured by the Particle Size Test Method.

Spheronized aggregates may be formed, for example, using a spheronization process. For example, fine drug particles may be dry mixed with one or more optional excipients. Following dry mixing, the agglomerates formed in the mixture can be stabilized. For example if the drug or excipient particles in the agglomerates contains amorphous content, the agglomerates can be exposed to moisture in humidified air to convert the amorphous content to stabilize the spheronized aggregates.

In some embodiments, the first sub-formulation, the second sub-formulation, or both may be formulated as engineered drug particles. Engineered drug particles are engineered to have a particular morphology. Examples of engineered drug particles include hollow shells, porous particles, wrinkled particles, and other geometries drug.

The sub-formulation configuration of the first sub-formulation and the second sub-formulation may be the same. It is understood that even if the sub-formulation configuration is the same, each sub-formulation may include one or more different excipients. The sub-formulation configuration of the first sub-formulation and the second sub-formulation may be different. It is understood that even if the sub-formulation configuration is different, each sub-formulation may include one or more of the same excipients. Table 1 shows possible sub-formulation configurations for the first sub-formulation and the second sub-formulation.

TABLE 1

| Sub-formulation 1 is configured as: | Sub-formulation 2 is configured as: |
|---|---|
| an ordered blend, | an ordered blend, |
| the ordered blend comprising: | the ordered blend comprising: |
| fine drug particles of the first drug; | fine drug particles of the second drug; |
| coarse carrier particles; | coarse carrier particles; |
| optionally a force control agent; and | optionally a force control agent; and |
| optionally fine excipient particles. | optionally fine excipient particles |
| an ordered blend, | combination particles, |
| the ordered blend comprising: | the combination particles comprising: |
| fine drug particles of the first drug; | the second drug; and |
| coarse carrier particles; | one or more excipients, |
| optionally a force control agent; and | wherein the one or more excipients |
| optionally fine excipient particles. | comprises a sugar, a biocompatible polymer, |
| | a phospholipid, a force control agent, or any |
| | combination thereof. |
| an ordered blend, | spheronized aggregates, |
| the ordered blend comprising: | the spheronized aggregates consisting of fine |
| fine drug particles of the first drug; | drug particles of the second drug. |
| coarse carrier particles; | |
| optionally a force control agent; and | |
| optionally fine excipient particles. | |
| an ordered blend, | spheronized aggregates, |
| the ordered blend comprising: | the spheronized aggregates comprising: |
| fine drug particles of the first drug; | fine drug particles of the second drug; and |
| coarse carrier particles; | fine excipient particles. |
| optionally a force control agent; and | |
| optionally fine excipient particles. | |
| an ordered blend, | engineered drug particles, |
| the ordered blend comprising: | the engineered drug particles consisting of |
| fine drug particles of the first drug; | crystalline second drug. |
| coarse carrier particles; | |
| optionally a force control agent; and | |
| optionally fine excipient particles. | |
| combination particles, | combination particles, |
| the combination particles comprising: | the combination particles comprising: |
| the first drug; and | the second drug; and |
| one or more excipients, | one or more excipients, |
| wherein the one or more excipients | wherein the one or more excipients |
| comprises sugar, a biocompatible polymer, a | comprises sugar, a biocompatible polymer, a |
| phospholipid, a force control agent, or any | phospholipid, a force control agent, or any |
| combination thereof. | combination thereof. |
| combination particles, | spheronized aggregates, |
| the combination particles comprising: | the spheronized aggregates consisting of fine |
| the first drug; and | drug particles of the second drug. |
| one or more excipients, | |
| wherein the one or more excipients | |
| comprises sugar, a biocompatible polymer, a | |
| phospholipid, a force control agent, or any | |
| combination thereof. | |
| combination particles, | spheronized aggregates, |
| the combination particles comprising: | the spheronized aggregates comprising: |
| the first drug; and | fine drug particles of the second drug; and |
| one or more excipients, | fine excipient particles. |
| wherein the one or more excipients | |
| comprises sugar, a biocompatible polymer, a | |
| phospholipid, a force control agent, or any | |
| combination thereof. | |
| combination particles, | engineered drug particles consisting of |
| the combination particles comprising: | crystalline second drug. |
| the first drug; and | |
| one or more excipients, | |
| wherein the one or more excipients | |
| comprises sugar, a biocompatible polymer, a | |
| phospholipid, a force control agent, or any | |
| combination thereof. | |
| spheronized aggregates, the spheronized | spheronized aggregates, the spheronized |
| aggregates consisting of fine drug particles | aggregates consisting of fine drug particles |
| of the first drug. | of the second drug. |
| spheronized aggregates, the spheronized | spheronized aggregates, the spheronized |
| aggregates consisting of fine drug particles | aggregates comprising: |
| of the first drug. | fine drug particles of the second drug; and |
| | fine excipient particles. |
| spheronized aggregates, the spheronized | engineered drug particles consisting of |
| aggregates consisting of fine drug particles | crystalline second drug. |
| of the first drug. | |
| spheronized aggregates, the spheronized | spheronized aggregates, |
| aggregates comprising: | the spheronized aggregates comprising: |
| fine drug particles of the first drug; and | fine drug particles of the second drug; and |
| fine excipient particles. | fine excipient particles. |
| spheronized aggregates, the spheronized | engineered drug particles consisting of |
| aggregates comprising: | crystalline second drug. |
| fine drug particles of the first drug; and | |
| fine excipient particles. | |
| engineered drug particles consisting of | engineered drug particles consisting of |
| crystalline first drug. | crystalline second drug. |

Upon aerosolization of a dry powder formulation, for example through the use of a dry powder inhaler, the aerodynamic particle size distribution (APSD) of the delivered drug can be characterized based on a mass median aerodynamic diameter (MMAD) of the drug containing particles. A drug containing particle is delivered from a sub-formulation and includes the drug of the sub-formulation from which it was delivered. For example, first drug containing particles are drug containing particles delivered from the first sub-formulation. Second drug containing particles are drug containing particles delivered from the second sub-formulation. Drug containing particles delivered from sub-formulation may be pure drug or include one or more excipients that were included in the sub-formulation.

The MMAD of the first drug delivered from the first sub-formulation may be different than the MMAD of the second drug delivered from the second sub-formulation. In some embodiments, upon aerosolization of the formulation the MMAD of the first drug and the MMAD of the second drug may each independently be 5 μm or less, 4 μm or less, 3 μm or less, 2.75 μm or less, 2.5 μm or less, 2.25 μm or less, 2 μm or less, 1.75 μm or less, 1.5 μm or less, 1.25 μm or less, 1 μm or less, or 0.5 μm or less as measured according to the aerodynamic particle size distribution (APSD) Method.

In some embodiments, the formulations of the present disclosure may be beneficial when combination therapy of two or more drugs having different target treatment locations within the respiratory tract is desired. Generally, the smaller the aerodynamic diameter, the deeper the drug particle may penetrate into the respiratory tract. As such, in some embodiments, it may be beneficial for the MMAD of the first drug delivered and the MMAD of the second drug to be different. In some embodiments, upon aerosolization of the formulation, the MMAD of the first drug and the second drug differ by 0.1 μm or greater, 0.2 μm or greater, 0.3 μm or greater, 0.4 μm or greater, 0.5 μm or greater, 0.6 μm or greater, 0.7 μm or greater, 0.8 μm or greater, 0.9 μm or greater, 1 μm or greater, 1.25 μm or greater, 1.5 μm or greater, or 1.75 μm or greater as measured according to the aerodynamic particle size distribution (APSD) Test Method. In some embodiments, the MMAD of the first drug and the second drug differ by 2 μm or less, 1.75 μm or less, 1.5 μm or less, 1.25 μm or less, 1 μm or less, 0.9 μm or less, 0.8 μm or less, 0.7 μm or less, 0.6 μm or less, 0.5 μm or less, 0.4 μm or less, 0.3 μm or less, or 0.2 μm or less as measured according to the APSD Test Method.

Upon aerosolization of a dry powdered formulation, drug containing particles have a fine particle fraction (FPF). FPF is a measurement of the fraction of delivered drug containing particles having an aerodynamic diameter of less than 5 μm FPF. FPF may be used to estimate how much of the drug is likely to be respirable. In some embodiments, upon aerosolization of the formulation, the first drug and the second drug each independently have an FPF of 20% or greater, 25% or greater, 30% or greater, 35% or greater, 40% or greater, 45% or greater, 50% or greater, 55% or greater, 60% or greater, 65% or greater, 70% or greater, or 75% or greater as measured according to the APSD Test Method. In some embodiments, upon aerosolization of the formulation, the first drug and the second drug each independently have an FPF of 80% or less, 75% or less, 70% or less, 65% or less, 60% or less, 55% or less, 50% or less, 45% or less, 40% or less, 35% or less, 30% or less or 25% or less as measured according to the APSD Test Method.

In some embodiments, it may be beneficial for the FPF of the first drug and the FPF of the second drug to be different. In some embodiments, the FPF of the first drug and the FPF of the second drug differ by 3% or greater, 5% or greater, 7% or greater, 10% or greater, 15% or greater, 20% or greater, 25% or greater, 30% or greater, or 35% or greater. In some embodiments, the FPF of the first drug and the FPF of the second drug differ by 40% or less, 35% or less, 30% or less, 25% or less, 20% or less, 15% or less, 10% or less, 7% or less, or 5% or less.

The first dry powder sub-formulation and the second dry powder sub-formulation are in contact prior to administration. For example, the first dry powder sub-formulation and the second dry powder sub-formulation are in contact prior to patient dose preparation of the dry powder inhaler. The first dry powder sub-formulation and the second dry powder sub-formulation are in contact prior to patient inhalation of a dry powder inhaler. The first dry powder sub-formulation and the second dry powder sub-formulation are in contact within a dose container configured for use in a dry powder inhaler. As such, the present disclosure provides dose containers having a formulation of the present disclosure disposed within and dry powder inhalers configured to administer the formulation from such dose container.

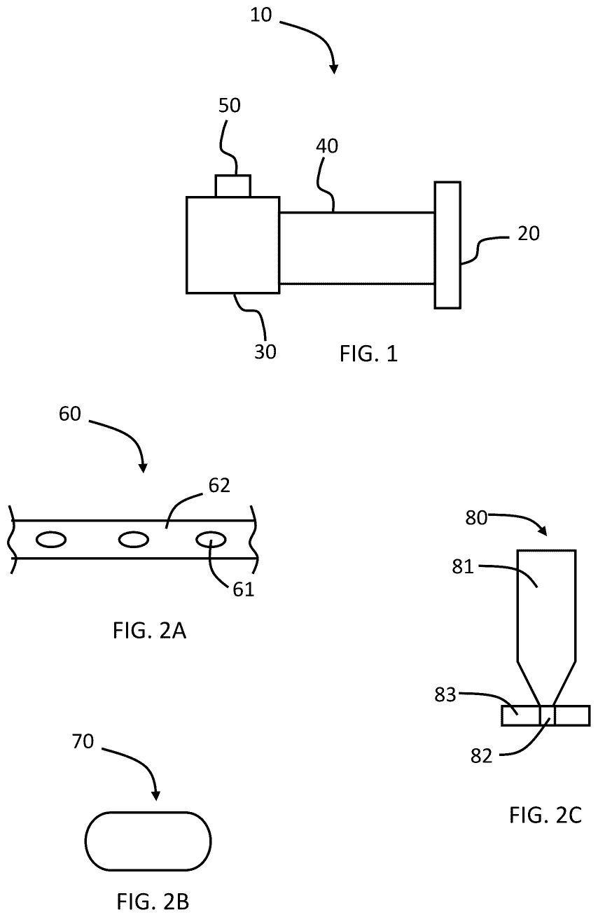

The dry powder inhaler formulation is configured for use in a dry powdered inhaler. Referring to FIG. 1, a dry powder inhaler 10 typically includes a dose storage compartment 30, a mouthpiece 20, a passageway 40, and a dose preparation mechanism 50. The passageway 40 fluidically couples the dose storage compartment 30 (and the dose container within the dose storage compartment) with the mouthpiece 20. The dose storage compartment 30 is configured to house a dose container. The dose preparation mechanism 50 is configured to prepare a single dose from the dose container for inhalation by the user. For example, the dose preparation mechanism is configured to release a single dose from a dose container. This may be done, for example, by peeling a blister to expose the powder formulation within the blister, by puncturing a capsule to expose the powder formulation in the capsule, or by metering out a single dose of powder formulation contained within a powder reservoir. When the dry powdered inhaler is used by a patient, the dose preparation mechanism allows a single dose to be fluidically coupled with the dose container and the passageway, and as the patient inhales, the dose travels through the passageway and exits the mouthpiece into the patient's mouth.

The dose storage compartment 30 is configured to house a dose container. A dose container is a receptacle that houses one or more doses of a single formulation (including a first sub-formulation in physical contact with a second sub-formulation), such as a formulation described herein. The dose storage compartment 30 may be configured to house one dose container. In some current combination dry powder inhaler therapies, two dose containers are included within the dose compartment, each dose container having a formulation that includes distinct drugs disposed within. During administration a dose from each dose container is released. In contrast, the present disclosure describes formulations that include sub-formulations where two drugs are administered simultaneously from a single dose container.

A dose container may contain a single dose or multiple doses. Dose containers that contain multiple doses may include single dose receptacles that hold individual doses. Alternatively, dose containers that contain multiple doses may hold two or more doses in physical contact. Examples of dose containers include blister strips, capsules, and reservoirs. In some embodiments, the dose container is in the form of a blister strip. In some embodiments, the dose container is in the form of a single blister. In some embodiments, the dose container is in the form of a capsule. In some embodiments, the dose container is in the form of a reservoir.

FIG. 2A shows a schematic of a portion of a blister strip 60. A blister strip typically includes multiple blisters 61 spaced along an elongated support tape 62. The blisters are single dose receptacles. The blisters are cavities formed between two strips of materials. Each blister contains a single dose. The formulation is disposed within the blister. As such, within a blister, the first sub-formulation and the second sub-formulation are in physical contact. Upon actuation, a blister may be pierced releasing the formulation or the two strips of material forming the blister may be peeled away from one another to release the formulation.

Some current combination therapies employ a dry powder inhaler having two blister strips, each blister strip containing a different drug formulation. Upon actuation, a blister from each blister strip releases the contained formulation. The formulations are mixed upon actuation and/or inhalation of the patient. As such, the formulations are not in physical contact prior to actuation of the inhaler. In contrast, the present disclosure provides a formulation having a first sub-formulation with a first drug and a second sub-formulation with a second drug where the first formulation and the second formulation are in physical contact before user actuation of the dose preparation mechanism and/or inhalation of the patient.

FIG. 2B shows a schematic of a capsule 70 dose container. A capsule is a container that holds a single dose. Upon actuation of the dose preparation mechanism the capsule is pierced or pulled apart to release the dose.

FIG. 2C shows a schematic of a reservoir 80 dose container. A reservoir is a container that holds multiple doses in contact with one another. Upon actuation of a dose preparation mechanism, a single dose is released. For example, a single dose is released from the reservoir into a holding container that is fluid communication with the passageway and mouthpiece.

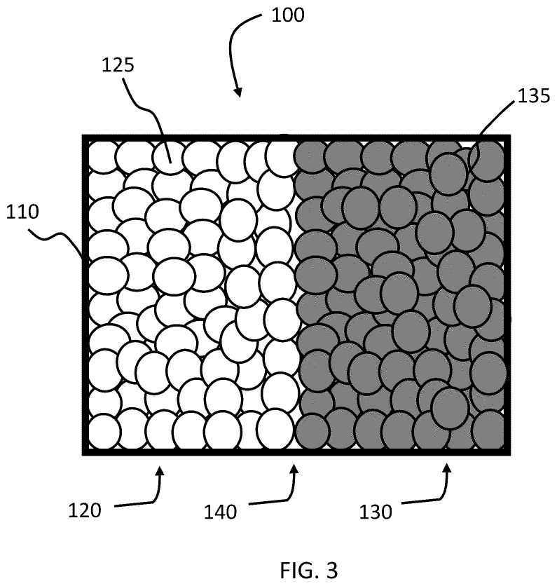

In the formulation, the first sub-formulation and the second sub-formulation are in physical contact. For example, within a dose container, at least a portion of the first sub-formulation is in physical contact with at least a portion of the second sub-formulation. In embodiments, where the dose container is a blister strip, the sub-formulations are in physical contact within each blister. The extent of intermixing and physical contact between portions of the sub-formulations may vary as schematically illustrated in FIGS. 3, 4, and 5. In some embodiments, the first sub-formulation and the second sub-formulation are not intermixed or only intermixed at an interface. In other embodiments, the first sub-formulation and the second sub-formulation are homogeneously or fully intermixed. In yet other embodiments, the first sub-formulation and the second sub-formulation are heterogeneously, or partially intermixed.

FIG. 3 is a schematic cross section of a dose or multiple doses of a formulation in which the first sub-formulation and the second sub-formulation are in contact but are not intermixed or only intermixed at an interface. The formulation 100 is disposed within a dose container containing a single dose (e.g., a capsule) or a dose receptacle (e.g., a blister of a blister strip) of a dose container (the barrier of the dose container or receptacle is indicated by element number 110). The formulation includes a first sub-formulation 125 and a second sub-formulation 135. Within the formulation, three regions exist. A first region 120 includes the first sub-formulation 125. The second region 130 includes the second sub-formulation 135. The fourth region 140 forms the interface between the first region 120 and the second region 130. At, or within the fourth region, the first sub-formulation is in contact with the second sub-formulation. The fourth region may include both the first sub-formulation and the second sub-formulation.

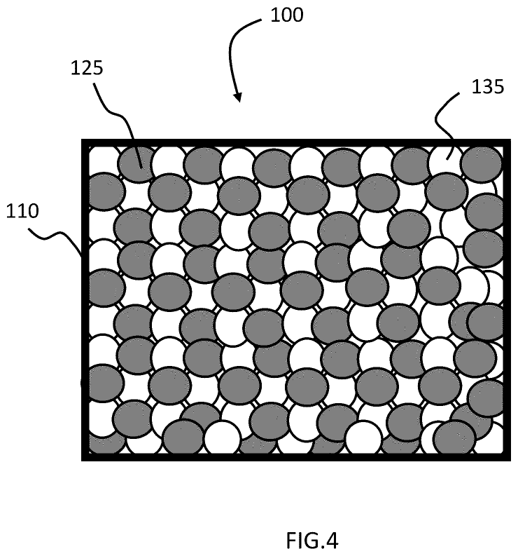

FIG. 4 is a schematic cross section of a dose or multiple doses of a formulation in which the first sub-formulation and the second sub-formulation are homogeneously or fully intermixed. The formulation 100 is disposed within a dose container (e.g., a reservoir or capsule) or a dose receptacle (e.g., a blister of a blister strip) of a dose container (the barrier of the dose container or receptable is indicated by element number 110). The formulation includes a first sub-formulation 125 and a second sub-formulation 135. The first sub-formulation and the second sub-formulation are blended such that no clear interface or regions of a single sub-formulation exist. As such, the formulation is a homogenous mixture of the first sub-formulation and the second sub-formulation.

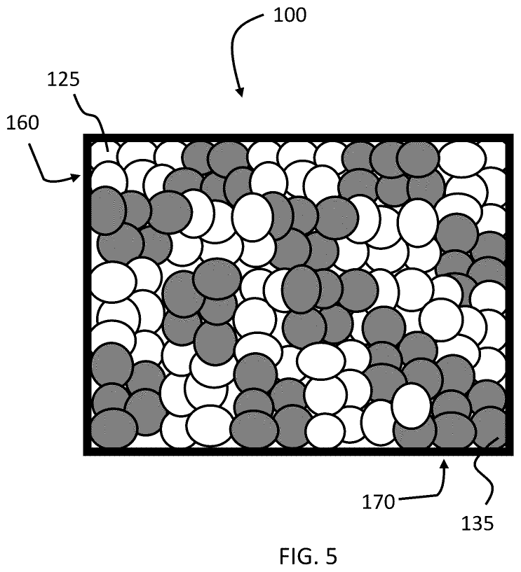

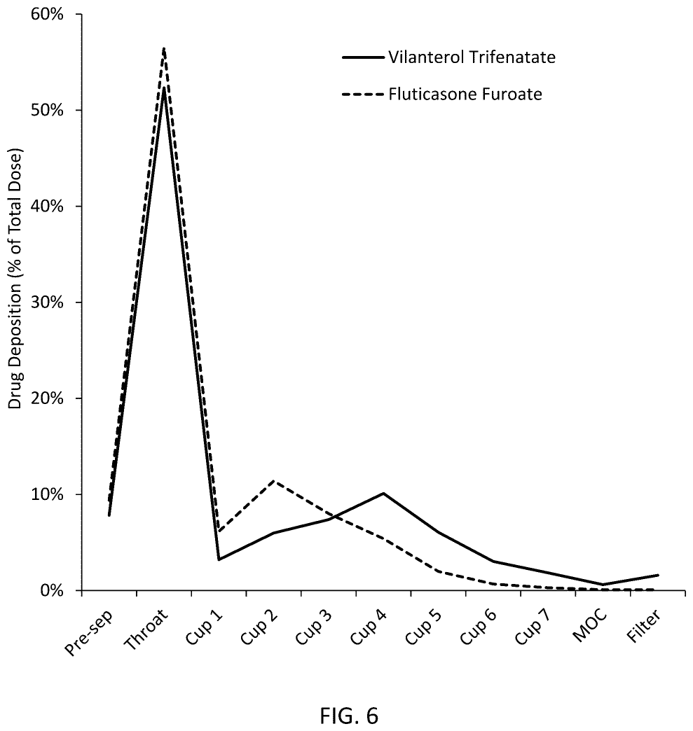

FIG. 5 is a schematic cross section of a dose or multiple doses of a formulation in which the first sub-formulation and the second sub-formulation are heterogeneously or partially intermixed. The formulation 100 is disposed within a dose container (e.g., a reservoir or capsule) or a dose receptacle (e.g., a blister of a blister strip) of a dose container (the barrier of the dose container or receptacle is indicated by the element number 10). The formulation includes a first sub-formulation 125 and a second sub-formulation 135. Within the formulation, a plurality of a first region 160 and a plurality of a second region 170 exists. Each first region 160 includes the first sub-formulation 125. Each second region 170 includes the second sub-formulation 135. The first regions and the second regions are distributed among the formulation such that the formulation has areas with a higher concentration of the first sub-formulation and areas having a higher concentration of the second sub-formulation. As such, the formulation is a heterogenous mixture of the first sub-formulation and the second sub-formulation.

The degree of intermixing can be influenced by mixing time, mixing speed, volume of each sub-formulation, the mixing equipment used, or a combination of these or other factors. The mixing time is the amount of time the first sub-formulation and the second sub-formulation were mixed together prior to disposing the resultant formulation in a dose container. A mixing time of 0 min indicates that the two sub-formulations were not mixed prior to placing in the dose container. For example, a mixing time of 0 min may result in a formulation where the first sub-formulation and the second sub-formulation are not intermixed or only intermixed at an interface. Generally, the greater the mixing time the more homogenous the first sub-formulation and second sub-formulation will be in a formulation.

Also provided are methods of making the formulations described herein, methods of making the dose containers containing the formulations described herein, and methods of making the dry powder inhalers described herein. All of the methods of making include contacting the first sub-formulation with the second sub-formulation. For example, the methods of making may include combining the first sub-formulation with the second sub-formulation such that they are in physical contact with each other.

In some embodiments, the method of making a formulation and the method of making a dose container containing the formulation and/or making the dry powder inhaler are the same. For example, in embodiments where the first sub-formulation and the second sub-formulation are in contact at an interface (not mixed, see FIG. 3) the formulation is formed by independently dispensing the first sub-formulation in the dose container and the second sub-formulation in the dose container.

The methods of making a dose container and/or making dry powdered inhaler of the present disclosure include dispensing the formulation into the dose container. The methods of making a dose container and/or making a dry powdered inhaler includes dispensing the formulation in a dose container such that the formulation is disposed within the dose container. In some embodiments where the dose container includes single dose receptables (e.g., a blister strip), the method includes dispensing the formulation into one or more single dose receptacle.

In some embodiments, prior to dispensing the formulation into the dose container, the first sub-formulation and the second sub-formulation are mixed. Stated differently, in some embodiments, the methods of making the formulation, making the dose container, and making the dry powder inhaler include mixing or blending the first sub-formulation with the second sub-formulation to form a formulation. Mixing of the sub-formulations may result in a formulation having a heterogenous or homogenous mixture of the first sub-formulation and the second sub-formulation (e.g., see FIG. 2 and FIG. 3). The mixing time, mixing speed, and mixing technique may depend at least in part on the desired homogeneity or heterogeneity of the resultant formulation.

When preparing bulk formulations for dispensing into two or more dose containers or receptables of a dose container (e.g., blisters of a blister strip), it may be desirable to minimize the amount of contact between the sub-formulations within the bulk formulation. However, at the same time, it may be desirable to have sufficient mixing of the sub-formulations in the bulk formulation such that each dose container and/or receptacle of a dose container (e.g., blisters of a blister strip) filled with the formulation has a similar amount of each sub-formulation to deliver a reproducible amount of each drug in every dose. For example, each blister of a blister strip may be filled with the formulation such that each blister delivers a similar amount of each drug in every dose (every blister).

Advantageously, dispensing a bulk formulation of the present disclosure into two or more dose containers may be simpler than separately dispensing two sub-formulations into each dose container. For example, a single dispensing instrument may be used.

Upon mixing, the uniformity of the formulation may be assessed. The uniformity of the formulation may be assessed, for example, using Raman chemical imaging. For example, a sample of a formulation is analyzed using microscopy combined with Raman spectroscopy. Alternatively, the degree of intermixing can be quantified using liquid chromatography according to the Formulation Uniformity Test Method.

The Formulation Uniformity Test Method includes sampling a volume of the formulation that is roughly equivalent to one half of the volume of formulation in a single dose at various positions within the volume of the bulk formulation and measuring the amount of each drug present in the sample using liquid chromatography. The percent relative standard deviation (% RSD) of each drug in the samples can be calculated and used as an approximate for formulation uniformity. For example, a formulation having two drugs, a first drug from a first sub-formulation and a second drug from a second sub-formulation, will have a first drug % RSD and a second rug % RSD. Generally, a lower % RSD indicates a higher formulation uniformity. It is understood, that % RSD is a measure of formulation uniformity across two or more doses. % RSD is not a measure of formulation uniformity within a single dose. In some embodiments, the method of forming the formulation results in a formulation having a first drug % RSD and a second drug % RSD that are each independently 20% or less, 15% or less, 10% or less, 5% or less, 2% or less, or 1% or less as measured according to the Formulation Uniformity Test Method.

### EXEMPLARY EMBODIMENTS

Embodiment 1 is a dry powder formulation configured for use with a dry powdered inhaler, the formulation comprising: a first sub-formulation comprising a first drug; and a second sub-formulation comprising a second drug, wherein the first drug and the second drug are different, and wherein the first sub-formulation and the second sub-formulation are in physical contact.

Embodiment 2a is the dry powder formulation of embodiment 1, wherein the first sub-formulation is excipient free, and the second formulation is excipient free.

Embodiment 2b is the dry powder formulation of embodiment 2a, wherein the first sub-formulation is configured as spheronized aggregates, the spheronized aggregates consisting of fine drug particles of the first drug.

Embodiment 2b is the dry powder formulation of embodiment 2a, wherein the first sub-formulation is configured as engineered particles, the engineered particles consisting of crystalline first drug.

Embodiment 2c is the dry powder formulation of embodiment 2b or 2c, wherein the second sub-formulation is configured as spheronized aggregates, the spheronized aggregates consisting of fine drug particles of the second drug.

Embodiment 2d is the dry powder formulation of embodiment 2b or 2c, wherein the second sub-formulation is configured as engineered particles, the engineered particles consisting of fine drug particles of the second drug.

Embodiment 3a is the dry powder formulation of embodiment 1, wherein the first sub-formulation is excipient free and the second sub-formulation comprises one or more excipients.

Embodiment 3b is the dry powder formulation of embodiment 3a, wherein the first sub-formulation is configured as spheronized aggregates, the spheronized aggregates consisting of fine drug particles of the first drug.

Embodiment 3c is the dry powder formulation of embodiment 3a, wherein the first sub-formulation is configured as engineered particles, the engineered particles consisting of crystalline first drug.

Embodiment 3d is the dry powder formulation of any one of embodiments 3a-3c, wherein the second sub-formulation comprises 0.01 wt-% to 30 wt-% second drug based on the weight of the second sub-formulation. In some embodiments, the second sub-formulation comprises 30 wt-% or less, 25 wt-% or less, 20 wt-% or less, 15 wt-% or less, 10 wt-% or less, 5 wt-% or less, 1 wt-% or less, 0.5 wt-% or less, or 0.1 wt-% or less of the second drug based on the weight of the second sub-formulation. In some embodiments, the second sub-formulation comprises 0.01 wt-% or greater, 0.1 wt-% or greater, 0.5 wt-% or greater, 1 wt-% or greater, 5 wt-% or greater, 10 wt-% or greater, 15 wt-% or greater, 20 wt-% or greater, or 25 wt-% or greater of the second based on the weight of the second sub-formulation.

Embodiment 3e is the dry powder formulation of any one of embodiments 3a-3d, wherein the second sub-formulation comprises 0.01 wt-% to 99.9 wt-% total excipients based on the weight of the second sub-formulation. In some embodiments, the second sub-formulation comprises 0.01 wt-% or greater, 0.1 wt-% or greater, 1 wt-% or greater, 10 wt-% or greater, 20 wt-% or greater, 30 wt-% or greater, 40 wt-% or greater, 50 wt-% or greater, 60 wt-% or greater, 70 wt-% or greater, 80 wt-% or greater, 90 wt-% or greater, or 95 wt-% or greater total excipients based on the weight of the second sub-formulation. In some embodiments, the second sub-formulation comprises 99.9 wt-% or less, 95 wt-% or less, 90 wt-% or less, 80 wt-% or less, 70 wt-% or less, 60 wt-% or less, 50 wt-% or less, 40 wt-% or less, 30 wt-% or less, 20 wt-% or less, 10 wt-% or less, 5 wt-% or less, 1 wt-% or less, or 0.1 wt-% or less total excipients based on the weight of the second sub-formulation.

Embodiment 3f is the dry powder formulation of any one of embodiment 3a-3e, wherein the one or more excipients comprise a sugar, a force control agent, a phospholipid, a biocompatible polymer, or any combination thereof.

Embodiment 3g is the dry powder formulation of embodiment 3f, wherein the sugar is present as fine excipient particles, coarse carrier particles, a force control agent, or any combination thereof.

Embodiment 3h is the dry powder formulation of embodiment 3f or 3g, wherein the sugar comprises lactose (e.g., lactose monohydrate), trehalose, sucrose, mannitol, or any combination thereof.

Embodiment 3i is the dry powder of formulation of any one of embodiments 3f to 3h, wherein the second sub-formulation comprises 0.01 wt-% to 99.9 wt-% total sugar based on the weight of the second sub-formulation.

Embodiment 3j is the dry powder formulation of any one of embodiments 3f to 3h, wherein the second sub-formulation comprises 40 wt-% to 99.5 wt-% coarse carrier particles based on the weight of the sub-formulation. In some embodiments, the second sub-formulation comprises 40 wt-% or greater, 50 wt-% or greater, 60 wt-% or greater, 70 wt-% or greater, 80 wt-% or greater, 90 wt-% or greater, or 95 wt-% or greater coarse carrier particles based on the weight of the second sub-formulation. In some embodiments, the second sub-formulation comprises 99.5 wt-% or less, 95 wt-% or less, 90 wt-% or less, 80 wt-% or less, 70 wt-% or less, 60 wt-% or less, or 50 wt-% or less coarse carrier particles based on the weight of the second sub-formulation.

Embodiment 3k is the dry powder formulation of any one of embodiments 3f to 3j, wherein the second sub-formulation comprises 0.1 wt-% to 90 wt-% fine excipient particles based on the weight of the second sub-formulation. In some embodiments, the second sub-formulation comprises 0.1 wt-% or greater, 1 wt-% or greater, 2 wt-% or greater, 3 wt-% or greater, 4 wt-% or greater, 5 wt-% or greater, 10 wt-% or greater, 15 wt-% or greater, or 20 wt-% or greater, 30 wt-% or greater, 40 wt-% or greater, 50 wt-% or greater, 60 wt-% or greater, 70 wt-% or greater, or 80 wt-% or greater fine excipient particles based on the weight of the second sub-formulation. In some embodiments, the second sub-formulation comprises 90 wt-% or less, 80 wt-% or less, 70 wt-% or less, 60 wt-% or less, 50 wt-% or less, 40 wt-% or less, 30 wt-% or less, 25 wt-% or less, 20 wt-% or less, 15 wt-% or less, 10 wt-% or less, 5 wt-% or less, 4 wt-% or less, 3 wt-% or less, 2 wt-% or less, or 1 wt-% or less fine excipient particles based on the weight of the second sub-formulation.

Embodiment 31 (i) is the dry powder formulation of any one of embodiments 3f to 3i, wherein the sugar is present as a force control agent.

Embodiment 31 (ii) is the dry powder formulation of embodiment 3f, wherein the biocompatible polymer is present as a force control agent. In some such embodiment, the biocompatible polymer comprises polyethylene glycol.

Embodiment 31 (iii) is the dry powder formulation of embodiment 3f, wherein force control agent comprises magnesium stearate, leucine (e.g., L-leucine), or both.

Embodiment 3m is the dry powder formulation of any one of embodiment 3f to 31, wherein the second sub-formulation comprises 0.1 wt-% to 5 wt-% of a force control agent based on the weight of the second sub-formulation. In some embodiments, the second sub-formulation comprises 0.1 wt-% or greater, 0.5 wt-% or greater, 0.7 wt-% or greater, 1 wt-% or greater, 2 wt-% or greater, 3 wt-% or greater, or 4 wt-% or greater of a force control agent based on the weight of the second sub-formulation. In some embodiments, the second sub-formulation 5 wt-% or less, 4 wt-% or less, 3 wt-% or less, 2 wt-% or less, 1 wt-% or less, 0.7 wt-% or less, or 0.5 wt-% or less of a force control agent based on the weight of the second sub-formulation.

Embodiment 3n is the dry powder formulation of embodiment 3f, wherein the phospholipid comprises a phosphatidylcholine. In some embodiments, the phosphatidylcholine comprises distearoylphosphatidylcholine, dipalmitoylphosphatidylcholine, or both.

Embodiment 30 is the dry powder formulation of embodiment 3f, wherein the biocompatible polymer comprises a natural polymer. In some embodiments, the natural polymer comprises albumin, carrageenan, chitosan, gelatin, hyaluronic acid, or any combination thereof.

Embodiment 3p is the dry powder formulation of embodiment 3f, wherein the biocompatible polymer comprises a synthetic polymer. In some embodiments, the synthetic polymer comprises poly(ethylene glycol), poly(lactic acid), oligo(lactic acid), poly(vinyl alcohol), acrylic acid derivatives, poly(lactic-co-glycolic acid), poly(lactic acid)-poly(ethylene glycol)-poly(lactic acid), or any combination thereof.

Embodiment 4a is the dry powder formulation of embodiment 1, wherein the first sub-formulation comprises one or more excipients and the second sub-formulation comprises one or more excipients.

Embodiment 4b is the dry powder formulation of embodiments 4a, wherein the first sub-formulation and the second sub-formulation each independently comprises 0.01 wt-% to 30 wt-% second drug based on the weight of the second sub-formulation. In some embodiments, the first sub-formulation and the second sub-formulation each independently comprise 30 wt-% or less, 25 wt-% or less, 20 wt-% or less, 15 wt-% or less, 10 wt-% or less, 5 wt-% or less, 1 wt-% or less, 0.5 wt-% or less, or 0.1 wt-% or less of the second drug based on the weight of the sub-formulation. In some embodiments, the first sub-formulation and the second sub-formulation each independently comprise 0.01 wt-% or greater, 0.1 wt-% or greater, 0.5 wt-% or greater, 1 wt-% or greater, 5 wt-% or greater, 10 wt-% or greater, 15 wt-% or greater, 20 wt-% or greater, or 25 wt-% or greater of the second based on the weight of the sub-formulation.

Embodiment 4c is the dry powder formulation of embodiments 4a or 4b, wherein the first sub-formulation and the second sub-formulation each independently comprises 0.01 wt-% to 99.9 wt-% total excipients based on the weight of the sub-formulation. In some embodiments, the first sub-formulation and the second sub-formulation each independently comprises 0.01 wt-% or greater, 0.1 wt-% or greater, 1 wt-% or greater, 10 wt-% or greater, 20 wt-% or greater, 30 wt-% or greater, 40 wt-% or greater, 50 wt-% or greater, 60 wt-% or greater, 70 wt-% or greater, 80 wt-% or greater, 90 wt-% or greater, or 95 wt-% or greater total excipients based on the weight of the sub-formulation. In some embodiments, the first sub-formulation and the second sub-formulation each independently comprises 99.9 wt-% or less, 95 wt-% or less, 90 wt-% or less, 80 wt-% or less, 70 wt-% or less, 60 wt-% or less, 50 wt-% or less, 40 wt-% or less, 30 wt-% or less, 20 wt-% or less, 10 wt-% or less, 5 wt-% or less, 1 wt-% or less, or 0.1 wt-% or less total excipients based on the weight of the sub-formulation.

Embodiment 4d is the dry powder formulation of any one of embodiment 4a-4c, wherein the one or more excipients comprise a sugar, a force control agent, a phospholipid, a biocompatible polymer, or any combination thereof.

Embodiment 4e is the dry powder formulation of embodiment 4d, wherein the sugar is present as fine excipient particles, coarse carrier particles, a force control agent, or any combination thereof.

Embodiment 4f is the dry powder formulation of embodiment 4d or 4e, wherein the sugar comprises lactose (e.g., lactose monohydrate), trehalose, sucrose, mannitol, or any combination thereof.

Embodiment 4g is the dry powder of formulation of any one of embodiments 4d to 4f, wherein the first sub-formulation and the second sub-formulation each independently comprises 0.01 wt-% to 99.9 wt-% total sugar based on the weight of the sub-formulation.

Embodiment 4h is the dry powder formulation of any one of embodiments 4d to 4g, wherein the first sub-formulation and the second sub-formulation each independently comprises 40 wt-% to 99.5 wt-% coarse carrier particles based on the weight of the sub-formulation. In some embodiments, the first sub-formulation and the second sub-formulation each independently comprises 40 wt-% or greater, 50 wt-% or greater, 60 wt-% or greater, 70 wt-% or greater, 80 wt-% or greater, 90 wt-% or greater, or 95 wt-% or greater coarse carrier particles based on the weight of the sub-formulation. In some embodiments, the first sub-formulation and the second sub-formulation each independently comprises 99.5 wt-% or less, 95 wt-% or less, 90 wt-% or less, 80 wt-% or less, 70 wt-% or less, 60 wt-% or less, or 50 wt-% or less coarse carrier particles based on the weight of the sub-formulation.

Embodiment 4i is the dry powder formulation of any one of embodiments 4d to 4h, wherein the first sub-formulation and the second sub-formulation each independently comprises 0.1 wt-% to 90 wt-% fine excipient particles based on the weight of the sub-formulation. In some embodiments, the first sub-formulation and the second sub-formulation each independently comprises 0.1 wt-% or greater, 1 wt-% or greater, 2 wt-% or greater, 3 wt-% or greater, 4 wt-% or greater, 5 wt-% or greater, 10 wt-% or greater, 15 wt-% or greater, or 20 wt-% or greater, 30 wt-% or greater, 40 wt-% or greater, 50 wt-% or greater, 60 wt-% or greater, 70 wt-% or greater, or 80 wt-% or greater fine excipient particles based on the weight of the sub-formulation. In some embodiments, the first sub-formulation and the second sub-formulation each independently comprises 90 wt-% or less, 80 wt-% or less, 70 wt-% or less, 60 wt-% or less, 50 wt-% or less, 40 wt-% or less, 30 wt-% or less, 25 wt-% or less, 20 wt-% or less, 15 wt-% or less, 10 wt-% or less, 5 wt-% or less, 4 wt-% or less, 3 wt-% or less, 2 wt-% or less, or 1 wt-% or less fine excipient particles based on the weight of the sub-formulation.

Embodiment 4j (i) is the dry powder formulation of any one of embodiments 4d-4g, wherein the sugar is present as a force control agent.

Embodiment 4j (ii) is the dry powder formulation of embodiment 4d, wherein the biocompatible polymer is present as a force control agent. In some such embodiment, the biocompatible polymer comprises polyethylene glycol.

Embodiment 4j (iii) is the dry powder formulation of embodiment 4d, wherein force control agent comprises magnesium stearate, leucine (e.g., L-leucine), or both.

Embodiment 4k is the dry powder formulation of any one of embodiment 4d to 4j, wherein the second sub-formulation comprises 0.1 wt-% to 5 wt-% of a force control agent based on the weight of the second sub-formulation. In some embodiments, the second sub-formulation comprises 0.1 wt-% or greater, 0.5 wt-% or greater, 0.7 wt-% or greater, 1 wt-% or greater, 2 wt-% or greater, 3 wt-% or greater, or 4 wt-% or greater of a force control agent based on the weight of the sub-formulation. In some embodiments, the first sub-formulation and the second sub-formulation may each independently include 5 wt-% or less, 4 wt-% or less, 3 wt-% or less, 2 wt-% or less, 1 wt-% or less, 0.7 wt-% or less, or 0.5 wt-% or less of a force control agent based on the weight of the sub-formulation

Embodiment 41 is the dry powder formulation of embodiment 4d, wherein the phospholipid comprises a phosphatidylcholine. In some embodiments, the phosphatidylcholine comprises distearoylphosphatidylcholine, dipalmitoylphosphatidylcholine, or both.

Embodiment 4m is the dry powder formulation of embodiment 4d, wherein the biocompatible polymer comprises a natural polymer. In some embodiments, the natural polymer comprises albumin, carrageenan, chitosan, gelatin, hyaluronic acid, or any combination thereof.

Embodiment 4n is the dry powder formulation of embodiment 4d, wherein the biocompatible polymer comprises a synthetic polymer. In some embodiments, the synthetic polymer comprises poly(ethylene glycol), poly(lactic acid), oligo(lactic acid), poly(vinyl alcohol), acrylic acid derivatives, poly(lactic-co-glycolic acid), poly(lactic acid)-poly(ethylene glycol)-poly(lactic acid), or any combination thereof.

Embodiments 5a-50 are each independently the dry powder formulation of embodiment 1 wherein the first sub-formulation and the second sub-formulation are configured as indicated in the table below and comprise the component indicated in the table below.

| Embodiment | First sub-formulation is | Second sub-formulation is |
|---|---|---|
| Number | configured as: | configured as: |
| 5a | an ordered blend, | an ordered blend, |
| | the ordered blend comprising: | the ordered blend comprising: |
| | fine drug particles of the first drug; | fine drug particles of the second |
| | coarse carrier particles; | drug; |
| | optionally a force control agent; and | coarse carrier particles; |
| | optionally fine excipient particles. | optionally a force control agent; and |
| | | optionally fine excipient particles |
| 5b | an ordered blend, | combination particles, |
| | the ordered blend comprising: | the combination particles |
| | fine drug particles of the first drug; | comprising: |
| | coarse carrier particles; | the second drug; and |
| | optionally a force control agent; and | one or more excipients, |
| | optionally fine excipient particles. | wherein the one or more excipients |
| | | comprises a sugar, a biocompatible |
| | | polymer, a phospholipid, a force |
| | | control agent, or any combination |
| | | thereof. |
| 5c | an ordered blend, | spheronized aggregates, |
| | the ordered blend comprising: | the spheronized aggregates |
| | fine drug particles of the first drug; | consisting of fine drug particles of |
| | coarse carrier particles; | the second drug. |
| | optionally a force control agent; and | |
| | optionally fine excipient particles. | |
| 5d | an ordered blend, | spheronized aggregates, |
| | the ordered blend comprising: | the spheronized aggregates |
| | fine drug particles of the first drug; | comprising: |
| | coarse carrier particles; | fine drug particles of the second |
| | optionally a force control agent; and | drug; and |
| | optionally fine excipient particles. | fine excipient particles. |
| 5e | an ordered blend, | engineered drug particles, |
| | the ordered blend comprising: | the engineered drug particles |
| | fine drug particles of the first drug; | consisting of second drug. |
| | coarse carrier particles; | |
| | optionally a force control agent; and | |
| | optionally fine excipient particles. | |
| 5f | combination particles, | combination particles, |
| | the combination particles | the combination particles |
| | comprising: | comprising: |
| | the first drug; and | the second drug; and |
| | one or more excipients, | one or more excipients, |
| | wherein the one or more excipients | wherein the one or more excipients |
| | comprises sugar, a biocompatible | comprises sugar, a biocompatible |
| | polymer, a phospholipid, a force | polymer, a phospholipid, a force |
| | control agent, or any combination | control agent, or any combination |
| | thereof. | thereof. |
| 5g | combination particles, | spheronized aggregates, |
| | the combination particles | the spheronized aggregates |
| | comprising: | consisting of fine drug particles of |
| | the first drug; and | the second drug. |
| | one or more excipients, | |
| | wherein the one or more excipients | |
| | comprises sugar, a biocompatible | |
| | polymer, a phospholipid, a force | |
| | control agent, or any combination | |
| | thereof. | |
| 5h | combination particles, | spheronized aggregates, |
| | the combination particles | the spheronized aggregates |
| | comprising: | comprising: |
| | the first drug; and | fine drug particles of the second |
| | one or more excipients, | drug; and |
| | wherein the one or more excipients | fine excipient particles. |
| | comprises sugar, a biocompatible | |
| | polymer, a phospholipid, a force | |
| | control agent, or any combination | |
| | thereof. | |
| 5i | combination particles, | engineered drug particles consisting |
| | the combination particles | of second drug. |
| | comprising: | |
| | the first drug; and | |
| | one or more excipients, | |
| | wherein the one or more excipients | |
| | comprises sugar, a biocompatible | |
| | polymer, a phospholipid, a force | |
| | control agent, or any combination | |
| | thereof. | |
| 5j | spheronized aggregates, the | spheronized aggregates, the |
| | spheronized aggregates consisting of | spheronized aggregates consisting of |
| | fine drug particles of the first drug. | fine drug particles of the second |
| | | drug. |
| 5k | spheronized aggregates, the | spheronized aggregates, the |
| | spheronized aggregates consisting of | spheronized aggregates comprising: |
| | fine drug particles of the first drug. | fine drug particles of the second |
| | | drug; and |
| | | fine excipient particles. |
| 5l | spheronized aggregates, the | engineered drug particles consisting |
| | spheronized aggregates consisting of | of second drug. |
| | fine drug particles of the first drug. | |
| 5m | spheronized aggregates, the | spheronized aggregates, |
| | spheronized aggregates comprising: | the spheronized aggregates |
| | fine drug particles of the first drug; | comprising: |
| | and | fine drug particles of the second |
| | fine excipient particles. | drug; and |
| | | fine excipient particles. |
| 5n | spheronized aggregates, the | engineered drug particles consisting |
| | spheronized aggregates comprising: | of second drug. |
| | fine drug particles of the first drug; | |
| | and | |
| | fine excipient particles. | |
| 5o | engineered drug particles consisting | engineered drug particles consisting |
| | of first drug. | of second drug. |

Embodiment 6a is the dry powder formulation of any one or embodiment 5a to 5o, wherein the fine excipient particles (if present), the coarse carrier particles (if present), a force control agent (if present) or any combination thereof comprise a sugar.

Embodiment 6b is the dry powder formulation of any one of embodiments 5a to 5o or 6a, wherein sugar (if present) comprises lactose (e.g., lactose monohydrate), trehalose, sucrose, mannitol, or any combination thereof.

Embodiment 6c is the dry powder of formulation of any one of embodiments 5a to 5o or 6a-6b, wherein the first sub-formulation and the second sub-formulation each independently comprises 0.01 wt-% to 99.9 wt-% total sugar (if present) based on the weight of the sub-formulation. In some embodiments, the first sub-formulation and the second sub-formulation each independently comprises 0.01 wt-% or greater, 0.1 wt-% or greater, 1 wt-% or greater, 10 wt-% or greater, 20 wt-% or greater, 30 wt-% or greater, 40 wt-% or greater, 50 wt-% or greater, 60 wt-% or greater, 70 wt-% or greater, 80 wt-% or greater, 90 wt-% or greater, or 95 wt-% or greater total sugar (if present) based on the weight of the sub-formulation. In some embodiments, the first sub-formulation and the second sub-formulation each independently comprises 99.9 wt-% or less, 95 wt-% or less, 90 wt-% or less, 80 wt-% or less, 70 wt-% or less, 60 wt-% or less, 50 wt-% or less, 40 wt-% or less, 30 wt-% or less, 20 wt-% or less, 10 wt-% or less, 5 wt-% or less, or 1 wt-% or less total sugar (if present) based on the weight of the sub-formulation.

Embodiment 6d is the dry powder formulation of any one of embodiments 5a to 50 or 6a-6c, wherein the first sub-formulation and the second sub-formulation each independently comprises 40 wt-% to 99. 5 wt-% coarse carrier particles (if present) based on the weight of the sub-formulation. In some embodiments, the first sub-formulation and the second sub-formulation each independent comprise 40 wt-% to 80 wt-% or 40 wt-% to 60 wt-% coarse carrier particles (if present) based on the weight of the sub-formulation.

Embodiment 6e is the dry powder formulation of any one of embodiments 5a to 50 or 6a to 6d, wherein the first sub-formulation and the second sub-formulation each independently comprises 0.1 wt-% or greater, 1 wt-% or greater, 2 wt-% or greater, 3 wt-% or greater, 4 wt-% or greater, 5 wt-% or greater, 10 wt-% or greater, 15 wt-% or greater, or 20 wt-% or greater, 30 wt-% or greater, 40 wt-% or greater, 50 wt-% or greater, 60 wt-% or greater, 70 wt-% or greater, or 80 wt-% or greater fine excipient particles (if present) based on the weight of the sub-formulation. In some embodiments, the first sub-formulation and the second sub-formulation each independently comprises 90 wt-% or less, 80 wt-% or less, 70 wt-% or less, 60 wt-% or less, 50 wt-% or less, 40 wt-% or less, 30 wt-% or less, 25 wt-% or less, 20 wt-% or less, 15 wt-% or less, 10 wt-% or less, 5 wt-% or less, 4 wt-% or less, 3 wt-% or less, 2 wt-% or less, or 1 wt-% or less fine excipient particles (if present) based on the weight of the sub-formulation. In some embodiments, the first sub-formulation and the second sub-formulation each independently comprise 1 wt-% to 30 wt-% fine excipient particles, such as 1 wt-% to 15 wt-% or 2 wt-% to 10 wt-% fine excipient particles (if present) based on the weight of the sub-formulation. In some embodiments, the first sub-formulation and the second sub-formulation each independently comprise 70 wt-% to 90 wt-% or 70 wt-% to 80 wt-% fine excipient particles (if present) based on the weight of the sub-formulation.

Embodiment 6f (i) is the dry powder formulation of any one of embodiments 5a to 50, or 6a-6c, wherein the sugar (if present) is present as force control agent.

Embodiment 6f (ii) is the dry powder formulation of any one of embodiments 5a to 50, wherein the biocompatible polymer (if present) is a force control agent. In some such embodiment, the biocompatible polymer comprises polyethylene glycol.

Embodiment 6f (iii) is the dry powder formulation of any one of embodiments 5a to 50, wherein force control agent comprises magnesium stearate, leucine (e.g., L-leucine), or both.

Embodiment 6g is the dry powder formulation of any one of embodiment 5a-50 or 6f, wherein the first sub-formulation and second-formulation each independently comprise 0.1 wt-% to 5 wt-% of a force control agent (if present) based on the weight of the sub-formulation. In some embodiments, first sub-formulation and second-formulation each independently comprise 0.5 wt-% to 2 wt-% of a force control agent (if present) based on the weight of the second sub-formulation. In some embodiments, the second sub-formulation comprises 0.5 wt-% to 1 wt-% of a force control agent (if present) based on the weight of the sub-formulation.

Embodiment 6h is the dry powder formulation of embodiment 5a-50, wherein the phospholipid (if present) comprises a phosphatidylcholine. In some embodiments, the phosphatidylcholine comprises distearoylphosphatidylcholine, dipalmitoylphosphatidylcholine, or both.

Embodiment 6i is the dry powder formulation of embodiment 5a-50, wherein the biocompatible polymer (if present) comprises a natural polymer. In some embodiments, the natural polymer comprises albumin, carrageenan, chitosan, gelatin, hyaluronic acid, or any combination thereof.

Embodiment 6j is the dry powder formulation of embodiment 5a-50, wherein the biocompatible polymer (if present) comprises a synthetic polymer of a synthetic block copolymer. In some embodiments, the synthetic polymer comprises poly(ethylene glycol), poly(lactic acid), oligo(lactic acid), poly(vinyl alcohol), acrylic acid derivatives, poly(lactic-co-glycolic acid), poly(lactic acid)-poly(ethylene glycol)-poly(lactic acid), or both.

Embodiment 7a is the dry powder formulation of any one of embodiments 1 to 61, wherein upon aerosolization of the dry powder formulation, the first drug and the second drug each independently have a MMAD of 5 μm or less, 4 μm or less, 3 μm or less, 2.75 μm or less, 2.5 μm or less, 2.25 μm or less, 2 μm or less, 1.75 μm or less, 1.5 μm or less, 1.25 μm or less, 1 μm or less, or 0.5 μm or less as measured according to the aerodynamic particle size distribution (APSD) Test Method.

Embodiment 7b is the dry powder formulation of any one of embodiments 1 to 7a, wherein upon aerosolization, the MMAD of the first drug and the MMAD of the second drug differ by 0.1 μm or greater, 0.2 μm or greater, 0.3 μm or greater, 0.4 μm or greater, 0.5 μm or greater, 0.6 μm or greater, 0.7 μm or greater, 0.8 μm or greater, 0.9 μm or greater, 1 μm or greater, 1.25 μm or greater, 1.5 μm or greater, or 1.75 μm or greater as measured according to the APSD Test Method. Upon aerosolization, the MMAD of the first drug and the MMAD of the second drug may differ by 2 μm or less, 1.75 μm or less, 1.5 μm or less, 1.25 μm or less, 1 μm or less, 0.9 μm or less, 0.8 μm or less, 0.7 μm or less, 0.6 μm or less, 0.5 μm or less, 0.4 μm or less, 0.3 μm or less, or 0.2 μm or less as measured according to the APSD Test Method.

Embodiment 7c is the dry powdered formulation of any one of embodiments 1 to 7b, wherein upon aerosolization of the formulation, the first drug and the second drug particles each independently have a fine particle fraction (FPF) of 20% or greater, 25% or greater, 30% or greater, 35% or greater, 40% or greater, 45% or greater, 50% or greater, 55% or greater, 60% or greater, 65% or greater, 70% or greater, or 75% or greater as measured according to the APSD Test Method. Upon aerosolization of the formulation, the first drug and the second drug may each independently have a FPF of 80% or less, 75% or less, 70% or less, 65% or less, 60% or less, 55% or less, 50% or less, 45% or less, 40% or less, 35% or less, 30% or less or 25% or less as measured according to the APSD Test Method.

Embodiment 7d is the dry powdered formulation of any one of embodiments 1 to 7c, wherein upon aerosolization of the formulation, the FPF of the first drug and the FPF of the second drug differ by 3% or greater, 5% or greater, 7% or greater, 10% or greater, 15% or greater, 20% or greater, 25% or greater, 30% or greater, or 35% or greater. Upon aerosolization of the formulation, the FPF of the first drug particles and the FPF of the second drug differ by 40% or less, 35% or less, 30% or less, 25% or less, 20% or less, 15% or less, 10% or less, 7% or less, or 5% or less.

Embodiment 8a is the dry powdered formulation of any one of embodiments 1 to 7d, wherein the first drug, the second drug, or both include a bronchodilator, an anti-inflammatory drug, or both.

Embodiment 8b is the dry powder formulation of embodiment 8a, wherein the bronchodilator includes a short-acting beta agonist, an intermediate-acting beta agonist, a long-acting beta agonist, an anticholinergic, a long-acting muscarinic agonist, or a methylxanthine.

Embodiment 8c is the dry powder formulation of embodiment 8b, wherein the short-acting beta agonist includes albuterol, levalbuterol, metaproterenol, terbutaline, epinephrine, a pharmaceutically acceptable salt thereof, a pharmaceutically acceptable ester thereof, or any combination thereof.

Embodiment 8d is the dry powder formulation of embodiment 8b, wherein the intermediate acting beta-agonist includes procaterol, a pharmaceutically acceptable salt thereof, a pharmaceutically acceptable ester thereof.

Embodiment 8e is the dry powder formulation of embodiment 8b, wherein the long-acting beta agonist includes abediterol, carmoterol, salmeterol, milveterol, formeoterol, indacaterol, arformoterol, vilanterol, olodaterol, a pharmaceutically acceptable salt thereof, a pharmaceutically acceptable ester thereof, or any combination thereof.

Embodiment 8f is the dry powder formulation of embodiment 8b, wherein the anticholinergic includes ipratropium, tiotropium, aclidinium, umeclidinium, oxitropium, glycopyrronium, a pharmaceutically acceptable salt thereof, a pharmaceutically acceptable ester thereof, or any combination thereof.

Embodiment 8g is the dry powder formulation of embodiment 8b, wherein the long-acting muscarinic agonist includes tiotropium, aclidinium, glycopyrronium, a pharmaceutically acceptable salt thereof, a pharmaceutically acceptable ester thereof, or any combination thereof.

Embodiment 8h is the dry powder formulation of embodiment 8b, wherein the methylxanthine includes theophylline, aminophylline, a pharmaceutically acceptable salt thereof, a pharmaceutically acceptable ester thereof, or any combination thereof.

Embodiment 8i is the dry powder formulation of embodiment 8a, wherein the anti-inflammatory drug includes a corticosteroid, an antihistamine, a mast cell stabilizer, or any combination thereof.

Embodiment 8j is the dry powder formulation of embodiment 8i, wherein the corticosteroid includes beclomethasone, budesonide, ciclesonide, fluticasone, flunisolide, triamcinolone, mometasone, a pharmaceutically acceptable salt thereof, a pharmaceutically acceptable ester thereof, or any combination thereof.

Embodiment 8k is the dry powder formulation of embodiment 8i, wherein the antihistamine includes cromolyn sodium, ketotifen, azelastine, a pharmaceutically acceptable salt thereof, a pharmaceutically acceptable ester thereof, or any combination thereof.

Embodiment 81 is the dry powder formulation of any one of embodiments 1 to 7d, wherein the first drug, the second drug, or both include omalizumab, zileuton, insulin, pentamidine, calcitonin, leuprolide, alpha-I-antitrypsin, interferon, nintedanib, a pharmaceutically acceptable salt thereof, a pharmaceutically acceptable ester thereof, or any combination thereof.

Embodiment 9a is the dry powder inhaler formulation of any one of embodiment 1 to 81, wherein, the first sub-formulation and the second sub-formulation are not intermixed or only intermixed at an interface.

Embodiment 9b is the dry powder inhaler formulation of any one of embodiment 1 to 81, wherein the first sub-formulation and the second sub-formulation are fully intermixed.

Embodiment 9c is the dry powder inhaler formulation of any one of embodiment 1 to 81, wherein the first sub-formulation and the second sub-formulation are partially intermixed.

Embodiment 10a is a dose container having the dry powder formulation of any one of embodiments 1 to 9c is disposed within the dose container, wherein the first sub-formulation and the second sub-formulation are in physical contact within the dose container.

Embodiment 10b is the dose container of embodiment 10a, wherein the dose container comprises a blister strip, a reservoir, or a capsule.

Embodiment 10c is the dose container of embodiment 10b, wherein the blister strip comprises a single dose receptacle and wherein the formulation is disposed within a single dose receptacle.

Embodiment 10c (i) is the dose container of embodiments 10c, wherein the first sub-formulation and the second sub-formulation are not intermixed or only intermixed at an interface within the single dose receptacle.

Embodiment 10c (ii) is the dose container of embodiments 10c, wherein the first sub-formulation and the second sub-formulation are fully intermixed within the single dose receptacle.

Embodiment 10c (iii) is the dose container of embodiments 10c, wherein the first sub-formulation and the second sub-formulation are partially intermixed within the single dose receptacle.

Embodiment 10e is the dose container of any one of embodiments 10a to 10b, wherein the first sub-formulation and the second sub-formulation are fully intermixed within the dose container.

Embodiment 10f is the dose container of any one of embodiments 10a to 10b, wherein the first sub-formulation and the second sub-formulation are partially intermixed within the dose container.

Embodiment 10d is the dose container of any one of embodiments 10a to 10b, wherein, the first sub-formulation and the second sub-formulation are not intermixed or only intermixed at an interface within the dose container.

Embodiment 10e is the dose container of any one of embodiments 10a to 10b, wherein the first sub-formulation and the second sub-formulation are fully intermixed within the dose container.

Embodiment 10f is the dose container of any one of embodiments 10a to 10b, wherein the first sub-formulation and the second sub-formulation are partially intermixed within the dose container.

Embodiment 11 is a dry powder inhaler comprising the dose container of any one of embodiment 10a to 10f.

Embodiment 12a is a method of making the dose container of any one of embodiments 10a-10f or the dry powder inhaler of embodiment 11, the method comprising dispensing the dry powder formulation into the dose container.

Embodiment 12b wherein the method comprises mixing the first sub-formulation with the second sub-formulation.

Embodiment 12c is the method of embodiment 12b, wherein mixing takes place prior to dispensing.

### EXAMPLES

These Examples are merely for illustrative purposes and are not meant to be overly limiting on the scope of the appended claims. Notwithstanding that the numerical ranges and parameters setting forth the broad scope of the present disclosure are approximations, the numerical values set forth in the specific Examples are reported as precisely as possible. Any numerical value, however, inherently contains certain uncertainty necessarily resulting from the standard deviation found in their respective testing measurements. At the very least, and not as an attempt to limit the application of the doctrine of equivalents to the scope of the claims, each numerical parameter should at least be construed in light of the number of reported significant digits and by applying ordinary rounding techniques.

Unless otherwise noted, all parts, percentages, ratios, etc. in the Examples and the rest of the specification are by weight.

The following abbreviations may be used in the following examples and/or other places in this disclosure: Mn=number average molecular weight; ppm=parts per million; ppb=parts per billion; mL=milliliter; L=liter; LPM=liters per minute; m=meter, mm=millimeter, min=minutes; s=seconds; cm=centimeter, μm=micrometer, kg=kilogram, g=gram, mcg or μg=microgram, mg=milligram, min=minute, s=second, h=hour,° C.=degrees Celsius,° F.=degrees Fahrenheit; wt-%=weight percent; M=molar; μM=micromole; mM=millimolar; and DI water=deionized water.

Table 2 is a materials table giving a list of components used in the Examples and their associated vendor source, abbreviation, and chemical abstract service (CAS) number.

TABLE 2
Materials used in the Examples.

| Material | Type of Reagent | Abbreviation | CAS No. |
|---|---|---|---|
| Vilanterol Trifenatate | Drug | VT | 503068-34-6 |
| Magnesium Stearate | Force Control | MgSt | 557-04-0 |
| | Agent | | |
| Lactose Monohydrate | Coarse Carrier | LCM | 10039-26-6 |
| | Particle/Fine | | |
| | excipient particles | | |
| Fluticasone Furoate | Drug | FTF | 397864-44-7 |
| Mometasone Furoate | Drug | MF | 83919-23-7 |

### Test Methods:

### Particle Size Test Method

The volume-weighted diameter at different metrics of any plurality of particles (e.g., coarse carrier particles, fine drug particles,) can be measured using various techniques including laser diffraction and optical microscopy. Laser diffraction methods can measure the particle size on drug powder that has either been dispersed in a suitable non-solvent liquid or has been dispersed in air. The overall particle size distribution can be characterized. Typical metrics include the volume-weighted diameter D₁₀ (particle size for which 10% of the total particle volume is contained in particles smaller than this value), volume-weighted media diameter D₅₀ (particle size for which 50% of the total particle volume is contained in particles smaller than this value), and similarly the volume-weighted diameter D₉₀, volume-weighted diameter D_{15.9}, and volume-weighted diameter D_{84.1}, the span (which is the D₉₀ minus D₁₀ divided by the D₅₀), and other particle size distribution summary statistics.

### Formulation Uniformity Test Method

The percent relative standard deviation of the drugs in the formulations was determined using the following technique. Following mixing the bulk formulation was transferred to a clean surface. A sample thief designed to capture about 12 mg or powder was used to obtain ten samples of formulation from different locations of the bulk formulation. The mass of each sample was gravimetrically measured. The mass of each drug in each sample was determined by dissolving the sample in diluent and measuring the amount of each drug present using liquid chromatography. The wt-% of each drug in each sample was calculated by dividing the mass of drug determined using liquid chromatography by the total sample weight. The % RSD for each drug in the formulation was calculated from the ten samples.

### Aerodynamic Particle Size Distribution (APSD) Test Method

APSD testing was conducted using the Next Generation Impactor (NGI) (TSI Incorporated, Shoreview, MN) at a flowrate of 100 liters per minute and a total flow volume of 5.8 L using the TOK 100i Critical Flow Controller (Copley Scientific, Nottingham, United Kingdom) and HCP6 High Capacity Pump (Copley Scientific). The United States Pharmacopeial inlet (USP Throat) and the NGI Pre-separator (TSI Incorporated) were utilized. The USP Throat and all NGI cups were coated using glycerol to prevent particle bounce. APSD testing consisted of delivery from a single capsule. Drug deposited in the capsule, DPI device and all stages, throat, pre-separator, and all NGI cups was dissolved using a diluent (e.g., 10 mM sodium dodecyl sulfate, 60:40 (v/v) acetonitrile: 50 mM ammonium acetate, pH 5.50) and analyzed by liquid chromatography.

The APSD summary statistics were calculated using CITDAS (Copley Scientific, Nottingham, United kingdom). Various metrics can be used to describe the APSD. For the examples presented, the APSD is described using the mass median aerodynamic diameter (MMAD; the aerodynamic particle size for which 50% of the total particle mass is contained in particles with an aerodynamic diameter smaller than this value), the geometric standard deviation, and the fine particle fraction (the fraction, presented as a percentage, of the dose emitted from the device with aerodynamic particle diameters less than or equal to 5.0 microns).

The APSD Test Method was used to calculate the APSD summary statistics of mass median aerodynamic diameter (MMAD), geometric standard deviation (GSD), and the fine particle fraction (FPF), after a formulation was delivered by the AEROLIZER dry powder inhaler. Additionally, the APSD Test Method was used to determine the average mass amount of the drug in the capsule (Caps.), the dry powder inhaler (DPI), US Pharmacopeia (USP) throat and decoupler, the next generation impactor (NGI) pre-separator, the micro orifice collector, and all of the NGI cups (i.e., cup 1, cup 2, cup 3, cup 4, cup 5, cup 6, and cup 7) after a formulation was delivered by the AEROLIZER dry powder inhaler.

### Example 1: Evaluation of Various Formulations Having a Vilanterol Trifenatate Sub-Formulation and a Fluticasone Furoate Sub-Formulation

The effect of sub-formulation mixing time on the ASPD properties of a formulation having a first sub-formulation containing vilanterol trifenatate (VT) and a second sub-formulation containing fluticasone furoate (FTF) was evaluated.

Table 3 shows the formulations tested in this Example. A VT control formulation (containing only VT, lactose and the FCA magnesium stearate) was tested. A FTF control formulation (containing only FTF and lactose) was also tested. For each VT control formulation sample and each FTF control formulation sample, 12.5 mg of the powdered formulation was loaded into a QUALI-V-I (QUALICAPS, Nara, Japan) capsule. For VF/FTF F1, approximately 12.5 mg of the sub-formulation vilanterol trifenatate (VT) was placed into the capsule and approximately 12.5 mg of the second sub-formulation containing fluticasone furoate (FTF) was added to the same capsule and the capsule was sealed prior to APSD testing. For each of VF/FTF F2 to F4, the sub-formulations were mixed for a mixing time using a Turbula Type T2F Shaker Mixer (VWR, Radnor, PA) at 57 RPM. Following mixing, approximately 25 mg of the resultant formulation was loaded into a QUALI-V-I (QUALICAPS, Nara, Japan) capsule. Three samples per control formulation and five samples per each VT/FTF formulation were tested according to the APSD Test Method. The % RSD of each drug in formulations F2-F4 as measured according to the Formulation Uniformity Test Method (see Table 4).

TABLE 3
Formulations tested in Example 1.

| | | | | Sub-formulation 2 | | | |
|---|---|---|---|---|---|---|---|
| | Sub-formulation 1 | | | | | | Mixing |
| | VT | FCA | LCM | FTF | FC | LCM | time |
| Formulation | (mcg) | (mcg) | (mg) | (mcg) | (mcg) | (mg) | (min) |
| VT Control | 40 | 125 | 12.34 | 0 | 0 | 0 | Not |
| | | | | | | | applicable |
| FTF Control | 0 | 0 | 0 | 200 | 0 | 12.3 | Not |
| | | | | | | | applicable |
| VT/FTF F1 | 40 | 125 | 12.34 | 200 | 0 | 12.3 | Not |
| | | | | | | | applicable |
| VT/FTF F2 | 1080 | 3375 | 333.18 | 5400 | 0 | 332.1 | 30 |
| VT/FTF F3 | 1080 | 3375 | 333.18 | 5400 | 0 | 332.1 | 10 |
| VT/FTF F4 | 1080 | 3375 | 333.18 | 5400 | 0 | 332.1 | 120 |

TABLE 4
% RSD of each drug in various formulation tested in Example 1

| | Mixing | | |
|---|---|---|---|
| Formulation | Time (min) | % RSD of VT | % RSD of FTF |
| VT/FTF F2 | 30 | 7.5% | 2.0% |
| VT/FTF F3 | 10 | 5.3% | 1.7% |
| VT/FTF F4 | 120 | 17.8% | 2.1% |

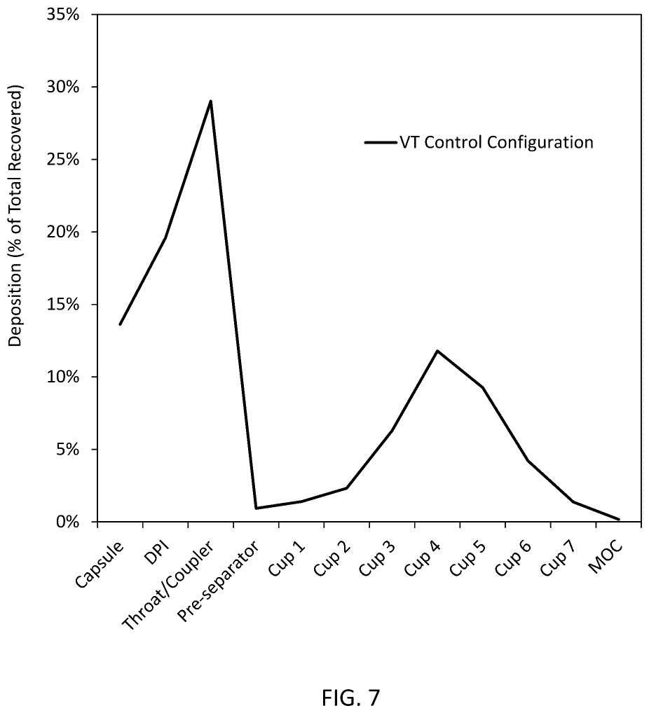

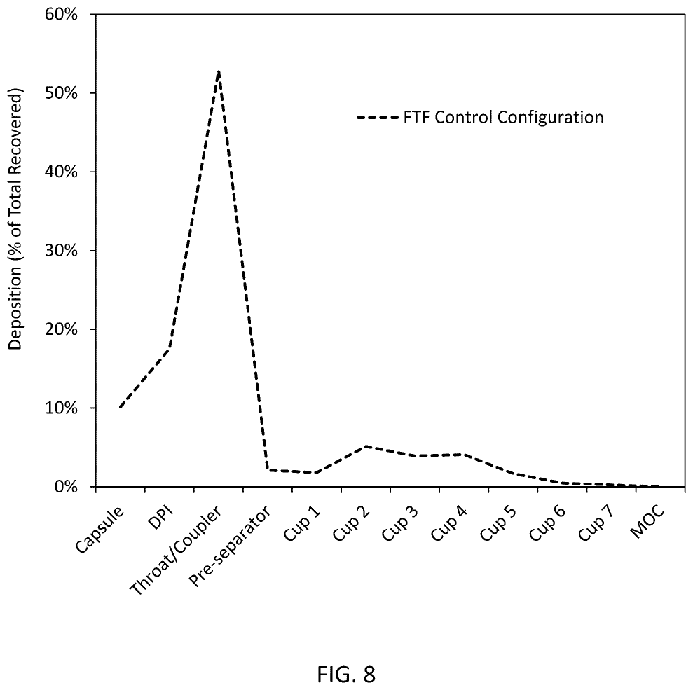

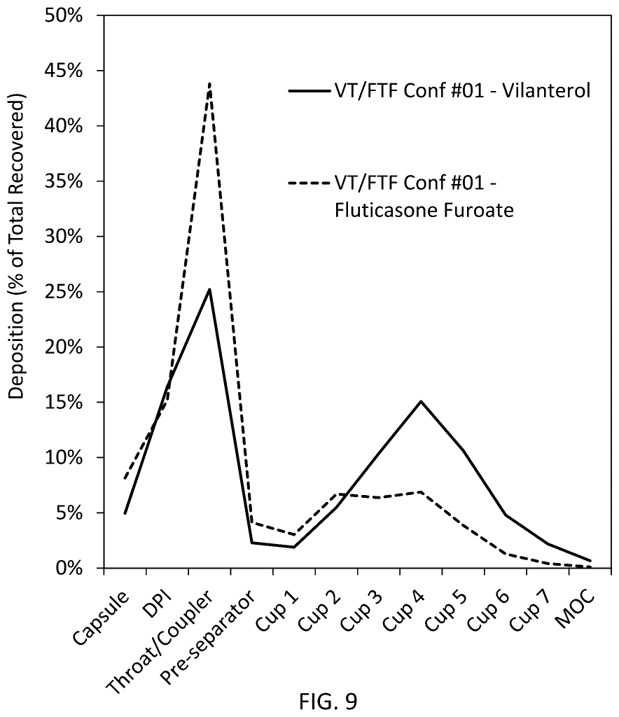

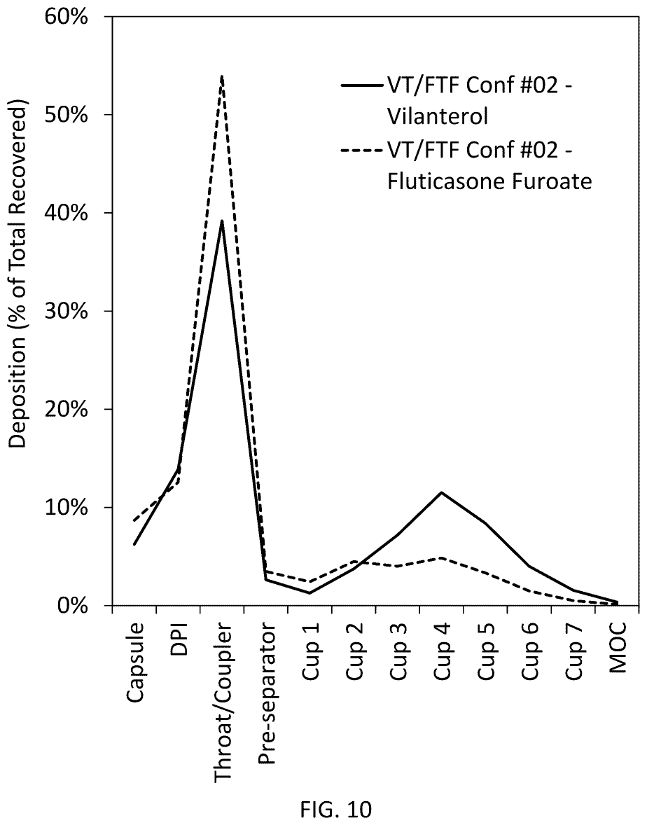

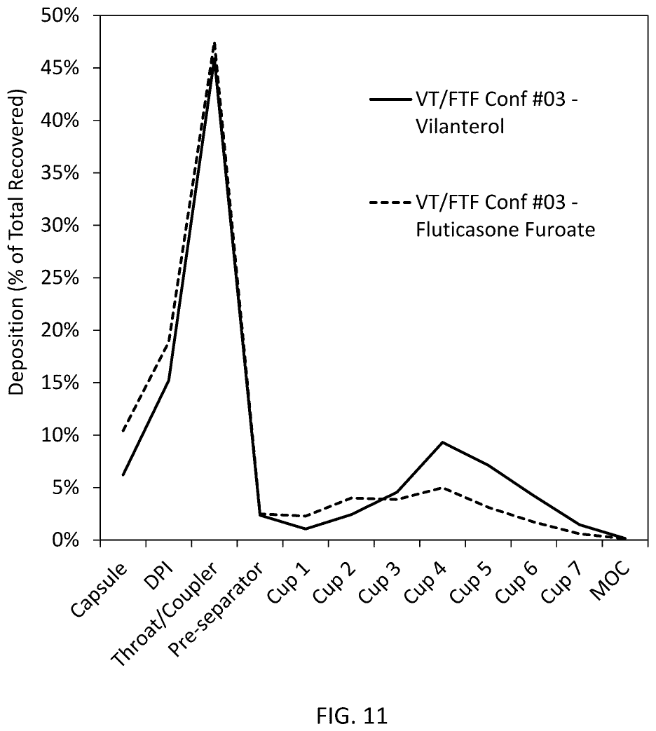

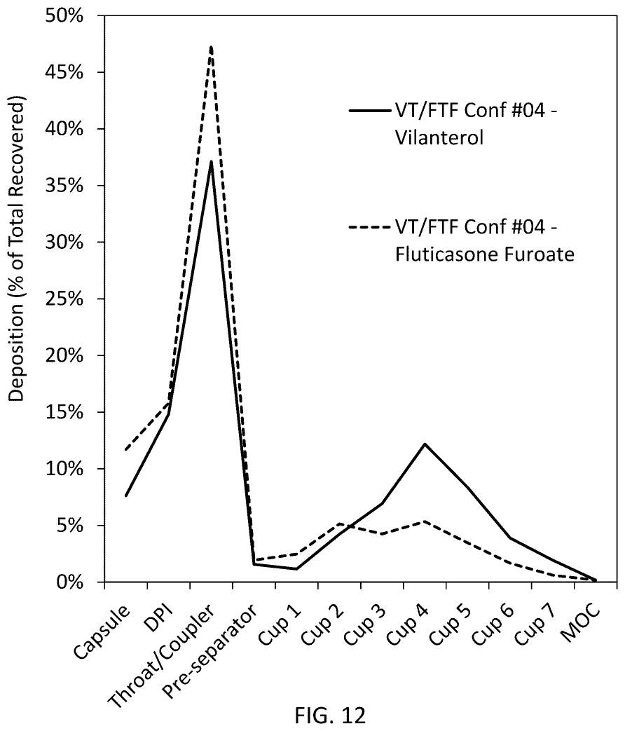

The average VT NGI profiles are shown in Table 5 and the average FTF NGI profiles are shown in Table 6. The APSD summary statistics are shown in Table 7. The average APSD profiles are shown in FIG. 7 (VT control), FIG. 8 (FTF control), FIG. 9 (VT/FTF F1), FIG. 10 (VT/FTF F2), FIG. 11 (VT/FTF F3), and FIG. 12 (VT/FTF F3). FIG. 7 and FIG. 8 demonstrate that the APSD from VT Control and FTF Control are significantly different from each other. The VT Control formulation, which includes a force control agent, has a higher FPF (%) and lower MMAD than does the FTF Control formulation. The VT and FTF APSD profiles were different from each other even when the two sub-formulations were delivered from the same capsule without mixing (FIG. 9) or with varying degrees of mixing (FIGS. 10-12).

TABLE 5
Average VT NGI Profiles

| | Average as % of Total Dose Delivered | | | | | | | | | | | |
|---|---|---|---|---|---|---|---|---|---|---|---|---|
| | | | USP | Pre- | Cup | | | | | | | |
| Formulation | Caps. | DPI | Throat | Sep. | 1 | 2 | 3 | 4 | 5 | 6 | 7 | MOC |
| VT Control | 13.60 | 19.60 | 29.00 | 0.90 | 1.40 | 2.30 | 6.30 | 11.80 | 9.30 | 4.20 | 1.40 | 0.20 |
| VT/FTF F1 | 5.00 | 16.40 | 25.20 | 2.30 | 1.90 | 5.50 | 10.40 | 15.10 | 10.60 | 4.80 | 2.20 | 0.70 |
| VT/FTF F2 | 6.20 | 13.90 | 39.20 | 2.60 | 1.30 | 3.70 | 7.20 | 11.50 | 8.40 | 4.00 | 1.60 | 0.40 |
| VT/FTF F3 | 6.20 | 15.20 | 45.90 | 2.40 | 1.10 | 2.40 | 4.60 | 9.30 | 7.10 | 4.20 | 1.50 | 0.20 |
| VT/FTF F4 | 7.60 | 14.80 | 37.10 | 1.60 | 1.20 | 4.20 | 6.90 | 12.20 | 8.40 | 3.90 | 1.90 | 0.20 |

TABLE 6
Average FTF NGI Profiles.

| | Average as % of Total Dose Delivered | | | | | | | | | | | |
|---|---|---|---|---|---|---|---|---|---|---|---|---|
| | | | USP | Pre- | Cup | | | | | | | |
| Formulation | Caps. | DPI | Throat | Sep. | 1 | 2 | 3 | 4 | 5 | 6 | 7 | MOC |
| FTF Control | 10.10 | 17.50 | 52.80 | 2.10 | 1.80 | 5.10 | 3.90 | 4.10 | 1.70 | 0.50 | 0.30 | 0.00 |
| VT/FTF F1 | 8.20 | 15.20 | 43.80 | 4.10 | 3.00 | 6.70 | 6.40 | 6.90 | 3.90 | 1.30 | 0.40 | 0.10 |
| VT/FTF F2 | 8.70 | 12.60 | 54.00 | 3.50 | 2.40 | 4.50 | 4.00 | 4.90 | 3.30 | 1.50 | 0.50 | 0.10 |
| VT/FTF F3 | 10.40 | 18.90 | 47.50 | 2.50 | 2.30 | 4.00 | 3.90 | 5.00 | 3.10 | 1.70 | 0.60 | 0.10 |
| VT/FTF F4 | 11.70 | 15.80 | 47.40 | 1.90 | 2.50 | 5.10 | 4.30 | 5.30 | 3.50 | 1.70 | 0.60 | 0.20 |

TABLE 7
APSD Summary Statistics.

| | VT | VT | | FTF | FTF | |
|---|---|---|---|---|---|---|
| | MMAD | GSD | VT | MMAD | GSD | FTF |
| Formulation | (μm) | (μm) | FPF | (μm) | (μm) | FPF |
| VT Control | 1.511 | 1.890 | 53.1% | — | — | — |
| FTF Control | — | — | — | 2.773 | 1.900 | 20.3% |
| VT/FTF F1 | 1.672 | 1.962 | 61.1% | 2.470 | 2.084 | 31.1% |
| VT/FTF F2 | 1.606 | 1.962 | 44.6% | 2.266 | 2.309 | 22.1% |
| VT/FTF F3 | 1.470 | 1.992 | 36.4% | 2.148 | 2.374 | 24.4% |
| VT/FTF F4 | 1.617 | 1.991 | 47.7% | 2.256 | 2.273 | 26.6% |

The VT fine particle fraction (FPF) was larger than the FTF FPF for all CT/FTF formulations. Additionally, the difference in fine particle fraction (FPF) of VT and FTF for each FVT/FTF formulation was greater than 10%.

The VT mass median aerodynamic diameter (MMAD) was smaller than the FPF MMAD for all VT/FTF formulations. Additionally, the difference in mass median aerodynamic diameter of VT and FTF for each FVT/FTF formulation was greater than 0.5 μm.

### Example 2: Evaluation of a Formulation Having a Vilanterol Trifenatate Sub-Formulation and a Mometasone Furoate Sub-Formulation

A formulation having a first sub-formulation containing vilanterol trifenatate (VT) and a second sub-formulation containing mometasone furoate (MF) was evaluated.

Table 8 shows the formulations tested in this Example. A MF control formulation was included. For each MF control formulation sample, approximately 6.5 mg of the powdered formulation was loaded into a QUALI-V-I (QUALICAPS, Nara, Japan) capsule. The MF control formulation consists of spheronized agglomerates consisting of approximately 14.67% (w/w) of mometasone furoate and 85.33% (w/w) of micronized lactose. For each VT/MF formulation sample 12.5 mg of the VT sub-formulation and 12.5 mg of the MF sub-formulation were loaded into a QUALI-V-I (QUALICAPS, Nara, Japan) capsule. Three samples for the MF Control formulation and three samples for the VT/MF formulation were tested according to the APSD Test Method.

TABLE 8
Formulations Tested in Example 2.

| | Sub-formulation 1 | | | Sub-formulation 2 | | |
|---|---|---|---|---|---|---|
| | VT | FCA | LCM | MF | FCA | LCM |
| Formulation | (mcg) | (mcg) | (mg) | (mcg) | (mcg) | (mg) |
| MF Control | 0 | 0 | 0 | 953 | 0 | 5547 |
| VT/MF F1 | 40 | 125 | 12.34 | 953 | 0 | 5547 |

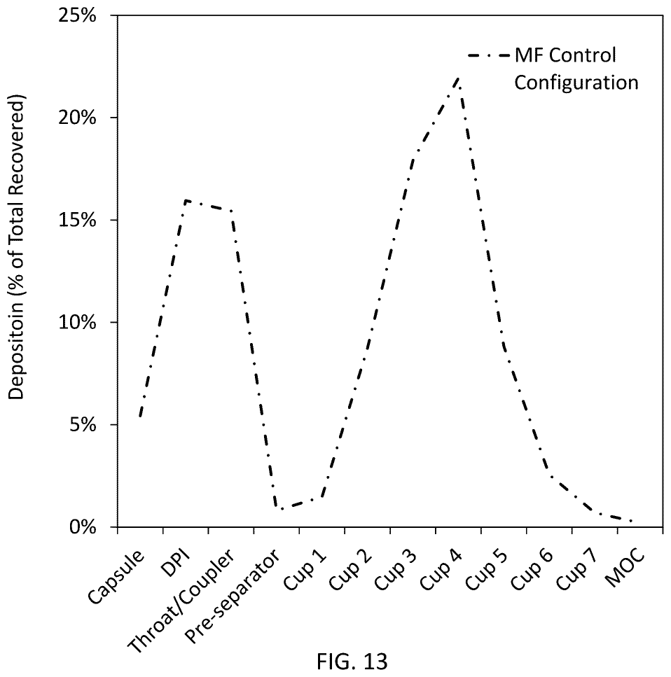

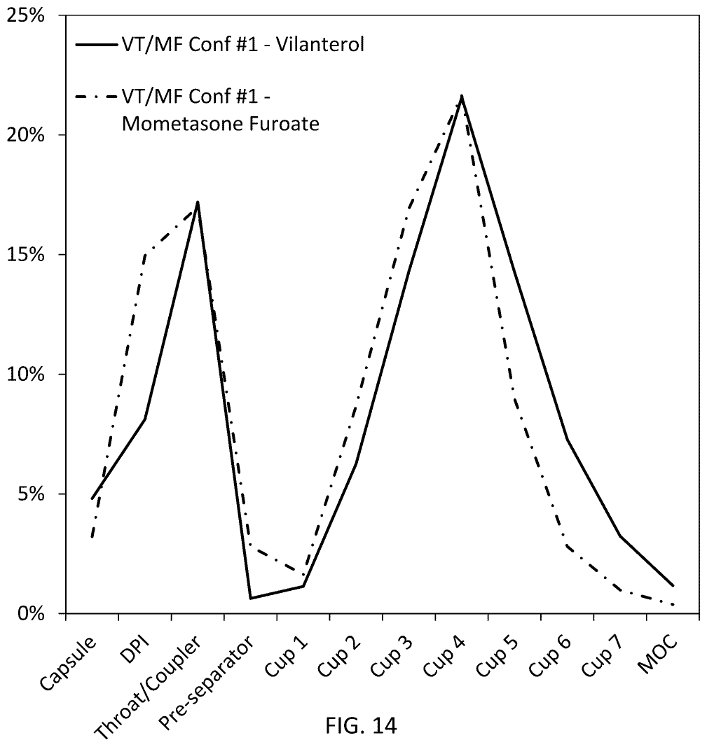

The average MF NGI profiles are shown in Table 9 and the average VT NGI profiles are shown in Table 10. The APSD summary statistics are shown in Table 11. The average APSD profiles are shown in FIG. 13 (MF control) and FIG. 14 (VT/MF F1). As seen in FIG. 14, the MF APSD is shifted to larger particle sizes relative to the VT APSD.

TABLE 9
Average MF NGI Profiles.

| | Average as % of Total Dose Delivered | | | | | | | | | | | |
|---|---|---|---|---|---|---|---|---|---|---|---|---|
| | | | USP | Pre- | Cup | | | | | | | |
| Formulation | Caps. | DPI | Throat | Sep. | 1 | 2 | 3 | 4 | 5 | 6 | 7 | MOC |
| MF Control | 5.40 | 15.90 | 15.40 | 0.80 | 1.50 | 8.70 | 17.90 | 21.90 | 8.80 | 2.60 | 0.70 | 0.20 |
| VT/MF F1 | 3.20 | 15.00 | 17.00 | 2.80 | 1.60 | 8.70 | 16.90 | 21.60 | 9.00 | 2.80 | 1.00 | 0.40 |

TABLE 10
Average VT NGI Profiles.

| | Average as % of Total Dose Delivered | | | | | | | | | | | |
|---|---|---|---|---|---|---|---|---|---|---|---|---|
| | | | USP | Pre- | Cup | | | | | | | |
| Formulation | Caps. | DPI | Throat | Sep. | 1 | 2 | 3 | 4 | 5 | 6 | 7 | MOC |
| VT/MF F1 | 4.80 | 8.10 | 17.20 | 0.60 | 1.10 | 6.30 | 14.30 | 21.60 | 14.30 | 7.30 | 3.20 | 1.20 |

TABLE 11
APSD Summary Statistics.

| | VT | VT | | MF | MF | |
|---|---|---|---|---|---|---|
| | MMAD | GSD | VT | MMAD | GSD | MF |
| Configuration | (μm) | (μm) | FPF | (μm) | (μm) | FPF |
| MF Control | — | — | — | 2.047 | 1.691 | 75.4% |
| VT/MF F1 | 1.601 | 1.850 | 76.7% | 2.012 | 1.732 | 71.7% |

The VT fine particle fraction (FPF) was larger than the MF FPF for the VT/MF formulation. The VT mass median aerodynamic diameter (MMAD) larger than MF MMAD for the VT/MF formulation.

All references and publications cited herein are expressly incorporated herein by reference in their entirety into this disclosure, except to the extent they may directly contradict this disclosure. Although specific embodiments have been illustrated and described herein, it will be appreciated by those of ordinary skill in the art that a variety of alternate and/or equivalent implementations can be substituted for the specific embodiments shown and described without departing from the scope of the present disclosure. It should be understood that this disclosure is not intended to be unduly limited by the illustrative embodiments and examples set forth herein and that such examples and embodiments are presented by way of example only with the scope of the disclosure intended to be limited only by the claims set forth here.

1. A dry powder inhaler comprising:
a dose container; and
a dry powder formulation contained in the dose container, the dry powder formulation comprising:
a first sub-formulation comprising a first drug; and
a second sub-formulation comprising a second drug;
the first sub-formulation and the second sub-formulation being in physical contact within the dose container; and
the first drug being different from the second drug.

2. The dry powder inhaler of claim 1, wherein:
the first sub-formulation is configured as spheronized aggregates consisting of fine particles of the first drug or the first sub-formulation is configured as engineered particles consisting of the first drug, and
the second sub-formulation is configured as spheronized aggregates consisting of fine particles of the second drug or the second sub-formulation is configured as engineered particles consisting of the second drug.

3. The dry powder inhaler of claim 1, wherein:
the first sub-formulation is configured as spheronized aggregates consisting of fine particles of the first drug or the first sub-formulation is configured as engineered particles consisting of the first drug, and
the second sub-formulation comprises one or more excipients.

4. The dry powder inhaler of claim 1, wherein the first sub-formulation comprises one or more excipients and the second sub-formulation comprises one or more excipients.

5. The dry powder inhaler of claim 1, wherein the one or more excipients comprises a sugar, a force control agent, a phospholipid, a biocompatible polymer, or any combination thereof.

6. The dry powder inhaler of claim 5, wherein the sugar is present as fine excipient particles, coarse carrier particles, a force control agent, or any combination thereof.

7. The dry powder inhaler of claim 5, wherein the sugar comprises lactose, trehalose, sucrose, mannitol, or any combination thereof.

8. The dry powder inhaler of claim 5, wherein the force control agent comprises magnesium stearate.

9. The dry powder inhaler of claim 5, wherein the phospholipid comprises a phosphatidylcholine.

10. The dry powder inhaler of claim 1, wherein the first drug, the second drug, or both comprise a bronchodilator drug, anti-inflammatory drug, or both.

11. The dry powder inhaler of claim 1, wherein the first sub-formulation and the second sub-formulation are not intermixed or only intermixed at an interface.

12. The dry powder inhaler of claim 1, wherein the first sub-formulation and the second sub-formulation are fully intermixed.

13. The dry powder inhaler of claim 1, wherein the first sub-formulation and the second sub-formulation are partially intermixed.

14. The dry powder inhaler of claim 1, wherein the dose container is in the form of a capsule or a reservoir.

15. The dry powder inhaler of claim 1, wherein the dose container is in the form of a blister strip and the formulation is disposed within one or more blisters along the blister strip.

16. The dry powder inhaler of claim 1, wherein upon aerosolization
the first drug and the second drug each independently have a mass median aerodynamic diameter (MMAD) of 5 μm or less as measured according to the aerodynamic particle size distribution (APSD) Test Method.

17. The dry powder inhaler claim 1, wherein upon aerosolization of the dry powder formulation,
the MMAD of the first drug and the MMAD of the second drug differ by 0.5 μm or greater as measured according to the APSD Test Method.

18. The dry powder inhaler of claim 1, wherein upon aerosolization of the dry powder formulation,
the first drug and the second drug each independently have a fine particle fraction (FPF) of 20% or greater as measured according to the APSD Test Method.

19. The dry powder inhaler of claim 1, wherein upon aerosolization of the dry powder formulation,
the FPF of the first drug and the FPF of the second drug differ by 10% or greater.

20. A method of making the dry powder inhaler of claim 1, wherein the method comprise dispensing the formulation into the dose container.
